(19) United States
(12) Patent Application Publication
(10) Pub. No.: US 2026/0216216 A1
(43) Pub. Date: Jul. 30, 2026

(54) (N-ALKYLDIHYDROL-2H-OXAZINYL)-CANNABIDIOL AS ANTI-PROLIFERATIVE AGENT AND PROCESS FOR PREPARATION THEREOF

(71) Applicant: COUNCIL OF SCIENTIFIC AND INDUSTRIAL RESEARCH, New Delhi (IN)

(72) Inventors: Pankaj Singh CHAM, Jammu (IN); Ajeet SINGH, Jammu (IN); Ashiya JAMWAL, Jammu (IN); Rattandeep SINGH, Jammu (IN); Shabu -, Jammu (IN); Sonia THAPA, Jammu (IN); Priya WAZIR, Jammu (IN); Utpal NANDI, Jammu (IN); Shashank Kumar SINGH, Jammu (IN); Parvinder Pal SINGH, Jammu (IN)

(21) Appl. No.: 19/141,661

(22) PCT Filed: Dec. 20, 2023
(86) PCT No.: PCT/IN2023/051204

§ 371 Date: Jun. 20, 2025

(30) Foreign Application Priority Data
Dec. 21, 2022 (IN) 202211074391

(51) Int. Cl. A61K 31/00; A61P 35/00; C07D 265/16; C07D 413/04; C07D 498/04

(52) U.S. Cl. CPC A61K 31/658; A61P 35/00; C07D 265/16; C07D 413/04; C07D 498/04

(57) ABSTRACT

The present invention relates to ring annulated analogues of cannabidiol where either one or both sides of the aromatic ring are attached to the oxazinyl ring, their method of preparation and use as anti-proliferative agents. The present invention provides a ring annulated cannabidiol analogue compound of Formula I. The present invention further provides a process for the synthesis of ring annulated cannabidiol analogue compound of Formula I.

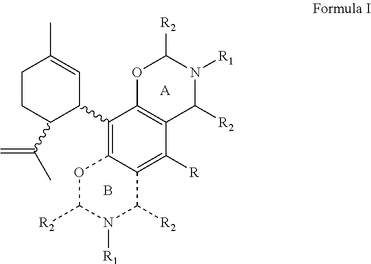

### FIELD OF INVENTION

The present invention relates to ring annulated analogues of cannabidiol having anti-proliferative property. The present invention particularly relates to ring annulated analogues of cannabidiol where either one or both sides of the aromatic ring are attached to the oxazinyl ring. The present invention also discloses their method of preparation and their use as anti-proliferative agents.

### BACKGROUND OF THE INVENTION

Cannabinoids either of endogenous or phytogenic or synthetic nature have been well known for their therapeutic potential in the management of pain, inflammation, nausea, vomiting and lack of appetite. However, last two decades research revealed that cannabinoids also have anti-proliferative potential against varieties of cancers (Cell Cycle, 2020, 19, 961; Cancers 2020, 12, 3203; Cancers 2022, 14(15), 3813; Curr. Pharm. Des., 2016, 22, 1756-1766).

Cannabinoids (endogenic, phytogenic and synthetic) explored for anti-proliferative properties, have been recently reviewed (Front Pharmacol., 2019, 10, 621; Trends Cancer., 2022, 8, 350-357).

Their anti-proliferative mechanism of action is not fully understood, however, several studies highlighted the role of several receptors and pathways which are implicated in cancer cell proliferation, migration, or death. The engagement of receptors and their downstream signal pathway depends upon the type of cannabinoids and cancers. The most notable key receptors responsible for the anti-proliferative potential are CB1, CB2 and GRP55 (G protein-coupled receptors), TRPV and TRPA (transient receptor potential channels), FAAH (fatty acid amide hydrolase), NAPE-PLD (N-acyl phosphatidylethanolamine phospholipase D), MAGL (monoacylglycerol lipase), DAGL (diacylglycerol lipase), ABHD6 (α/β-hydrolase domain containing 6), or ABHD12 (α/β-hydrolase domain containing 12) etc. (Br. J. Cancer., 2022, 127, 1-13).

Phytocannabinoids namely Δ⁹-tetrahydrocannabinol (Δ⁹-THC) and cannabidiol (CBD) has been well studied for anti-proliferative properties. Apart from these, other phytocannabinoids studied for anti-proliferative properties are Δ⁹-tetrahydrocannabinolic acid (Δ⁹-THCA), cannabidiolic acid (CBDA), cannabigerol (CBG), and cannabichromene (CBC), cannabinol (CBN) (Front. Pharmacol., 2019, 10, 621).

Δ⁹-Tetrahydrocannabinol (Δ⁹-THC) has shown anti-proliferative activity against glioblastoma, breast cancer, oral cancer, lung cancer, lymphoma, and leukaemia however, on the other hand, cannabidiol (CBD) has shown activity against glioblastoma, leukaemia, lung cancer, breast cancer, cervical cancer, prostate cancer, and melanoma. Moreover, it has been seen that cannabinoids enriched fractions have shown better effect than pure phytocannabinoids, which has been observed in some clinical trials, however, the size of those trials is still small, therefore more efforts are required to fully exploit the use of cannabinoids in clinical use (Br. J. Cancer., 2022, 127, 1-13).

Moreover, low oral bio-availability and poor-drug properties are some of the other concerns associated with natural phytocannabinoids halted their progress (Front Pharmacol., 2018, 9, 1-28; Br. J. Clin. Pharmacol., 2018, 84 (11), 2477-2482).

In addition to natural, synthetic phytocannabinoids namely dexanabinol also shown anti-cancer activity and is also being studied in advanced preclinical stages for the potential treatment of solid tumours (NCT01489826). Apart from these, several semi-synthetic and synthetic cannabinoids such as CP-55,940, WIN55,212-2, JWH-015, JWH-133, SR141716 (rimonabant), SR144528, and ACEA are also 25 being explored (Clin. Pharmacol. Ther., 2015, 97, 553-558; Molecules 2021, 26(6), 1761).

All these data showed that cannabinoids present good potential as anti-proliferative agents; however, still more efforts are required to have next generation candidate with better profile, target engagement and drug-likeness. Cannabidiol being non-psychotic is always the best choice for further exploration, and in this direction, the present invention disclosed the novel ring annulated analogues of cannabidiol as anti-proliferative agents.

WO2021/062559, describes the carboxamide-based cannabidiol derivatives and WO2021/062557, describes the carboxamide-based tetrahydrocannabinol derivatives.

WO2021/000054, describes the dioxinonyl-based cannabinoid derivatives for the treatment and prevention of diseases such as acute pain, ADHD/ADD, alcohol use disorder, allergic asthma, ALS, Alzheimer's, anorexia etc. In WO2021/000054, the synthesis required cannabidiolic acid (CBDA) as starting material.

However, the ring annulated analogues of cannabidiol of the present invention, differs with respect to the oxazinyl ring on one or both the side of the aromatic ring of cannabidiol and has anti-proliferative activity.

### OBJECTIVE OF THE INVENTION

The main object of the present invention is to provide the novel ring annulated analogues of cannabidiol.

Another object of the present invention is to provide a process for the synthesis of these analogues of cannabidiol by using mannich reaction such as a primary amine.

Yet another object of the present invention is to provide ring annulated analogues of CBD as anti-cancer agents.

### BRIEF DESCRIPTION OF THE DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts representation of a) dose-response curve of Id against different cancer cell lines; b IC₅₀ values of Id against different cancer cell lines, in accordance with an embodiment of the present disclosure.

### LIST OF ABBREVIATIONS

A549—Lung Cancer
CBD—Cannabidiol
CD₃OD—Deuterated Methanol
CDCl₃—Deuterated Chloroform
CH₃OH—Methanol
CHCl₃—Chloroform
DEPT—Distortionless Enhancement by Polarization Transfer
H₂O—Water
HRMS—High Resolution Mass Spectrometry
HCHO—Formaldehyde
HCT-116—Colorectal Cancer
HEK-293—Normal Cell Lines
IC₅₀—Inhibitory Concentration
LC-MS—Liquid Chromatogram-Mass Spectrometry
MS—Mass Spectrometry
MHz—Megahertz
MeOH—Methanol
MCF-7—Breast Cancer
Mia-PaCa—Pancreatic Cancer
nm—Nanometre
NMR—Nuclear Magnetic Resonance
NCI—National Cancer Institute
O.D.—Optical Density
PPM—Parts Per Million
PC-3—Prostate Cancer
RBF—Round Bottom Flask
RPMI—Roswell Park Memorial Institute
SRB—Sulphorhodamine
TLC—Thin Layer Chromatography
TOF—Turnover Frequency
Tris—tris(hydroxymethyl)aminomethane
TCA—Trichloroacetic acid
UHD—Ultra high definition
U251—Glioblastoma

### SUMMARY OF THE INVENTION

Accordingly, the present invention provides a ring annulated cannabidiol analogue compound of Formula I

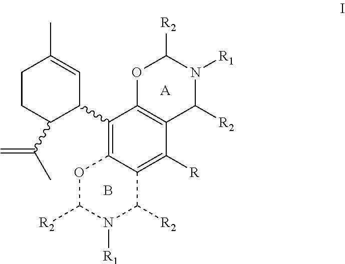

wherein, both A and B rings are simultaneously present or at least one of the ring is present;
R is selected from H, OH, protected hydroxyl, alkyl, alkenyl, alkynyl, acyl, aryl, heteroaryl, cycloalkyl and heterocyclyl, wherein the alkyl, alkenyl, alkynyl and acyl group is optionally substituted with one or more groups, independently selected from the group consisting of halogen, —F, —F₂, —F₃, —Br, —Br₂, —Br₃, Cl, —Cl₂, —Cl₃, —I, —I₂, —I₃, —OH, alkyl, —O-alkyl, NR′R″, S-alkyl, —SO-alkyl, —SO₂-alkyl, S-aryl, —SO-aryl, —SO₂-aryl, —SO₂—N-aryl, —N—SO₂-arylalkenyl, alkynyl, aryl, heteroaryl, cycloalkyl or heterocyclyl and the aryl or heteroaryl group is optionally substituted with one or more substituents selected from the group consisting of halogen, —OH, alkyl, —O-alkyl, —COOH, —C(O), —C alkyl, —C(O)OC and NR′R″;
R₁ is independently selected from H, alkyl, alkenyl, alkynyl, acyl, aryl, heteroaryl, cycloalkyl, or heterocyclyl, wherein the alkyl, alkenyl, alkynyl, aryl and acyl group is optionally substituted with one or more groups, independently selected from the group consisting of halogen, —F, —F₂, —F₃, —Br, —Br₂, —Br₃, Cl, —Cl₂, —Cl₃, —I, —I₂, —I₃, —OH, alkyl, —O-alkyl, —O-haloalkyl, NR′R″, S-alkyl, —SO-alkyl, —SO₂-alkyl, S-aryl, —SO-aryl, —SO₂-aryl, —SO₂—N-aryl, —N—SO₂-arylalkenyl, alkynyl, aryl, heteroaryl, cycloalkyl or heterocyclyl and the aryl or heteroaryl group is optionally substituted with one or more substituents selected from the group consisting of halogen, OH, alkyl, —O-alkyl, —COOH, —C(O), —C alkyl, —C(O)OC, NR′R″;
R₂ is selected from H, alkyl, alkyl aryl and aryl;
R′ and R″ are independently selected from the group consisting of H, alkyl, alkenyl, alkynyl or acyl.

In a preferred embodiment of the present invention R₁ in the compound of formula I is selected from the group consisting of:
methyl ethyl n-propyl n-butyl n-hexyl

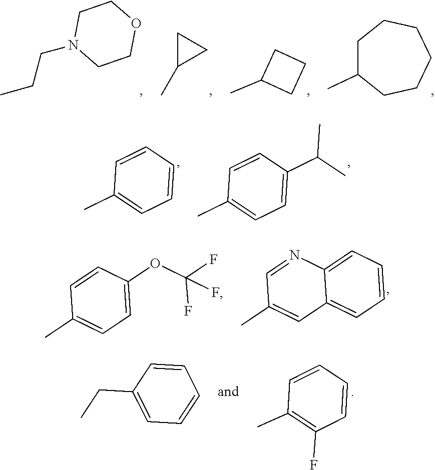

In a preferred embodiment of the present invention cannabidiol analogue is selected from the group consisting of (N-methyldihydrol-2H-oxazinyl)-cannabidiol, (N-ethyldihydrol-2H-oxazinyl)-cannabidiol, (N-propyldihydrol-2H-oxazinyl)-cannabidiol, (N-butyldihydrol-2H-oxazinyl)-cannabidiol, bis-{(N-butyldihydrol-2H-oxazinyl)]}-cannabidiol, (N-hexyldihydrol-2H-oxazinyl)-cannabidiol, (N-morpholinopropyldihydrol-2H-oxazinyl)-cannabidiol, (N-cyclopropyldihydrol-2H-oxazinyl)-cannabidiol, (N-cyclobutyldihydrol-2H-oxazinyl)-cannabidiol, (N-cycloheptyldihydrol-2H-oxazinyl)-cannabidiol, (N-phenyldihydrol-2H-oxazinyl)-cannabidiol, (N-isopropylphenyldihydrol-2H-oxazinyl)-cannabidiol, (N-trifluoromethoxyphenyldihydrol-2H-oxazinyl)-cannabidiol, (N-quinolinedihydrol-2H-oxazinyl)-cannabidiol, (N-benzyldihydrol-2H-oxazinyl)-cannabidiol and (N-2-fluorophenyldihydrol-2H-oxazinyl)-cannabidiol.

The present invention provides a process for the synthesis of ring annulated cannabidiol analogue compound of Formula I

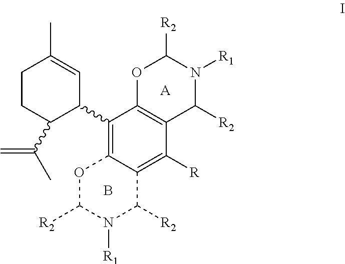

wherein, both A and B rings are simultaneously present or at least one of the ring is present;
R is selected from H, OH, protected hydroxyl, alkyl, alkenyl, alkynyl, acyl, aryl, heteroaryl, cycloalkyl and heterocyclyl, wherein the alkyl, alkenyl, alkynyl and acyl group is optionally substituted with one or more groups, independently selected from the group consisting of halogen, —F, —F₂, —F₃, —Br, —Br₂, —Br₃, Cl, —Cl₂, —Cl₃, —I, —I₂, —I₃, —OH, alkyl, —O-alkyl, NR′R″, S-alkyl, —SO-alkyl, —SO₂-alkyl, S-aryl, —SO-aryl, —SO₂-aryl, —SO₂—N-aryl, —N—SO₂-arylalkenyl, alkynyl, aryl, heteroaryl, cycloalkyl or heterocyclyl and the aryl or heteroaryl group is optionally substituted with one or more substituents selected from the group consisting of halogen, —OH, alkyl, —O-alkyl, —COOH, —C(O), —C alkyl, —C(O)OC, and NR′R″;
R₁ is independently selected from H, alkyl, alkenyl, alkynyl, acyl, aryl, heteroaryl, cycloalkyl, or heterocyclyl, wherein the alkyl, alkenyl, alkynyl, aryl, and acyl group is optionally substituted with one or more groups, independently selected from the group consisting of halogen, —F, —F₂, —F₃, —Br, —Br₂, —Br₃, Cl, —Cl₂, —Cl₃, —I, —I₂, —I₃, —OH, alkyl, —O-alkyl, —O-haloalkyl, NR′R″, S-alkyl, —SO-alkyl, —SO₂-alkyl, S-aryl, —SO-aryl, —SO₂-aryl, —SO₂—N-aryl, —N—SO₂-arylalkenyl, alkynyl, aryl, heteroaryl, cycloalkyl or heterocyclyl and the aryl or heteroaryl group is optionally substituted with one or more substituents selected from the group consisting of halogen, OH, alkyl, —O-alkyl, —COOH, —C(O), —C alkyl, —C(O)OC, NR′R″;
R₂ is selected from H, alkyl, alkyl aryl and aryl;
R′ and R″ are independently selected from the group consisting of H, alkyl, alkenyl, alkynyl or acyl, comprising the steps of:
i. coupling of fragment II wherein R is as defined above and fragment III wherein R₁ is as defined above in the presence of an aldehyde IV wherein R₂ is as defined above, at a temperature in the range of 25-30° C., for a period in the range of 13-16 hours optionally in presence of a solvent and

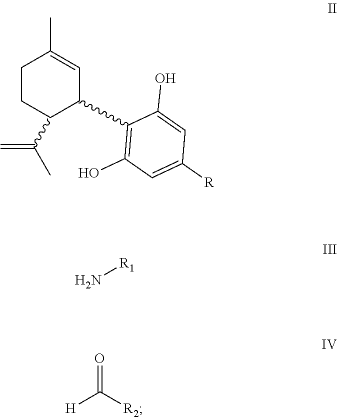

ii. purifying the obtained compound of formula I.

In an embodiment of the present invention the solvent used in the process is selected from the group consisting of dichloromethane, chloroform, isopropanol, acetone, acetonitrile methanol or a combination thereof.

In a preferred embodiment of the present invention fragment III is selected from the group consisting of:

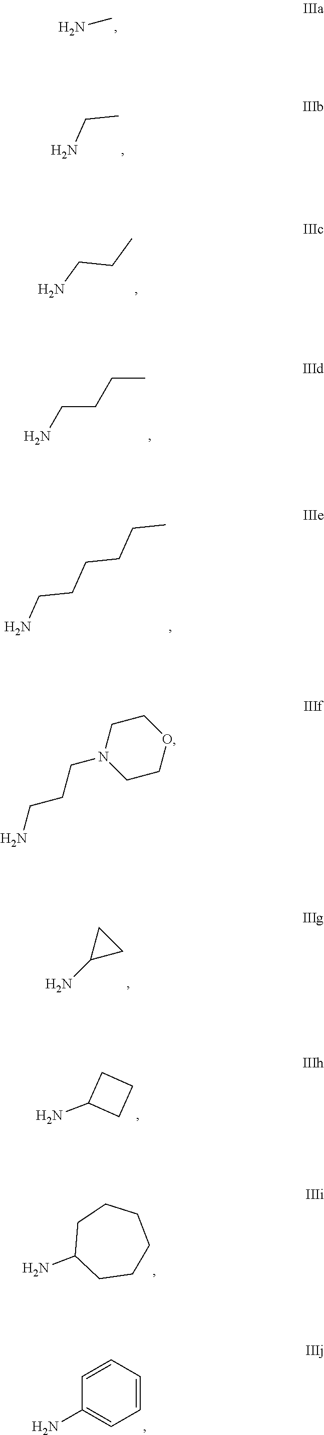

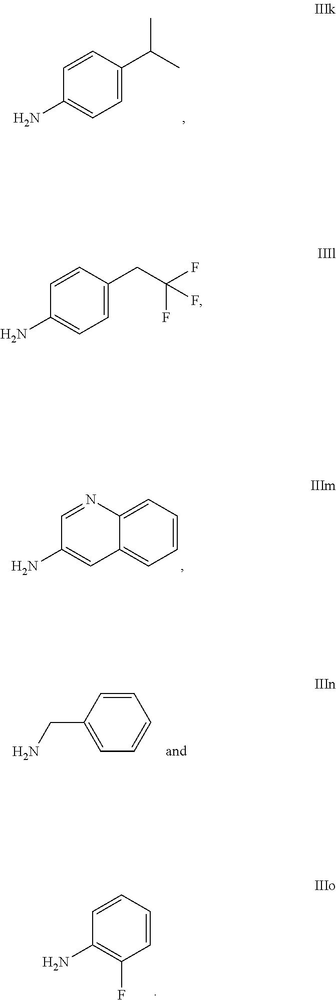

In a preferred embodiment of the present invention the aldehyde IV is selected from the group consisting of formaldehyde, propionaldehyde, acetaldehydes, propionaldehyde, butyraldehyde, isovaleraldehyde, benzaldehyde and cinnamaldehyde.

The cannabidiol analogue of the present invention has two stereo-centers which may be R or S as well as mixture of both.

In an embodiment of the present invention the compounds are useful as anti-proliferative agents and the compound exhibits in vitro anti-proliferative activity against colorectal (HCT-116), breast (MCF-7), lung (A549), pancreatic (Mia-PaCa), prostate (PC-3) and glioblastoma (U251) cancers.

In another embodiment of the present invention, the compounds of formula A exhibits in vitro anti-proliferative activity against cancer lines HCT-116, MCF-7, A549, Mia-PaCa, PC-3, and U251 representing colorectal cancer, breast cancer, lung cancer, pancreatic cancer, prostate cancer, and glioblastoma cell lines.

### DETAILED DESCRIPTION OF THE INVENTION

For a better understanding of the invention, a detailed description of the invention along with detailed description of the preferred embodiments of the present invention is explained below with reference to the accompanying tables and drawings.

For better understanding the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “consisting of means the embodiment necessarily includes the listed components only and no other unlisted components are present.

As used herein, the term “substituted” is contemplated to include all permissible substituents of organic compounds. In a broad aspect, the permissible substituents include acyclic and cyclic, branched, and unbranched, carbocyclic, and heterocyclic, aromatic, and nonaromatic substituents of organic compounds. Illustrative substituents, for example, include those described herein above. The permissible substituents can be one or more and the same or different for appropriate organic compounds. For purposes of this invention, the heteroatoms such as nitrogen may have hydrogen substituents, and/or any permissible substituents of organic compounds described herein which satisfy the valences of the heteroatoms. It is understood that the substituent may be further substituted.

The term “alkyl” refers to straight or branched aliphatic hydrocarbon groups having the specified number of carbon atoms, which are attached to the rest of the molecule by a single atom, which may be optionally substituted by one or more substituents. Preferred alkyl groups include, without limitation, methyl, ethyl, n-propyl, isopropyl, butyl, isobutyl, t-butyl, pentyl, hexyl, heptyl, octyl and the like.

The term “O-alkoxy” refers to an alkyl group attached via an oxygen linkage to the rest of the molecule, which may be optionally substituted by one or more substituents. Alkoxy groups refer to compounds with 1 to 10 carbon atoms and preferred alkoxy groups include, without limitation, —OCH₃, —OC₂H₅ and the like.

The term “halo” or “halogen” alone or in combination with other term(s) means fluorine, chlorine, bromine or iodine.

The term “haloalkyl” refers to alkyl with one or more halogen atoms. In the present invention, the term haloalkyl refers to compounds with 1 to 10 carbon atoms and examples of haloalkyl includes but not limited to —CH₂F, —CHF₂, —CF₃, —C₂H₄F and the like.

The term “haloalkoxy” refers to alkoxy group with one or more halogen atoms. The one or more hydrogens of the alkoxy group is substituted with one or more halogens. Examples of haloalkoxy group includes but not limited to —OCF₃, —OCH₂F, and the like.

The term “aryl” refers to aromatic radicals having 6 to 10 carbon atoms, which may be optionally substituted by one or more substituents. Preferred aryl groups include not limited to phenyl and the like.

The term “heteroaryl” refers to an aromatic heterocyclic ring radical as defined above. The term “heteroatom” as used herein designates a sulfur, nitrogen or oxygen atom. The heteroaryl ring radical may be attached to the main structure at any heteroatom or carbon atom resulting in the creation of a stable structure. The heteroaryl refers to an aromatic ring with one or more hetero atoms selected from N, O or S with carbon ranging between 2 to 10.

The term “heterocyclyl” refers to a heterocyclic ring radical that may be optionally substituted by one or more substituents. The heterocyclyl ring radical may be attached to the main structure at any heteroatom or carbon atom resulting in the creation of a stable structure. Furthermore, the term “heterocyclyl” refers to a stable 3 to 20 membered rings radical, which consists of carbon atoms and heteroatoms selected from nitrogen, phosphorus, oxygen and sulfur. For purposes of this invention the heterocyclic ring radical may be monocyclic, bicyclic or tricyclic ring systems, and the nitrogen, phosphorus, carbon, or sulfur atoms in the heterocyclic ring radical may be optionally oxidized to various oxidation states. In addition, the nitrogen atom may be optionally quaternized; and the ring radical may be partially or fully saturated. Preferred heterocyclyl groups include, without limitation, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, qunioxalinyl, quinolinyl, isoquinolinyl, tetrazolyl, imidazolyl, tetrahydroisoquinolinyl, piperidinyl, piperazinyl, homopiperazinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, triazolyl, indanyl, isoxazolyl, isoxazolidinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, thienyl, morpholinyl, thiomorpholinyl, thiamorpholinyl sulfoxide, furyl, tetrahydrofuryl, tetrahydropyranyl, chromanyl, and isochromanyl. The term “heterocyclyl” refers to monocyclic or polycyclic ring, polycyclic ring system refers to a ring system containing 2 or more rings, preferably bicyclic or tricyclic rings, in which rings can be fused, bridged or spiro rings or any combinations thereof. A fused ring as used herein means that the two rings are linked to each other through two adjacent ring atoms common to both rings. The fused ring can contain 1-4 hetero atoms independently selected from N, O, or S. The rings can be either fused by nitrogen or —CH— group.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. The term “between” should be understood as being inclusive of the limits.

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively and any and all combinations of any or more of such steps or features.

The present invention provides a ring annulated cannabidiol analogue compound of Formula I

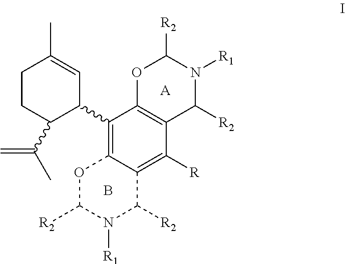

wherein, both A and B rings are simultaneously present or at least one of the ring is present;
R is selected from H, OH, protected hydroxyl, alkyl, alkenyl, alkynyl, acyl, aryl, heteroaryl, cycloalkyl and heterocyclyl, wherein the alkyl, alkenyl, alkynyl and acyl group is optionally substituted with one or more groups, independently selected from the group consisting of halogen, —F, —F₂, —F₃, —Br, —Br₂, —Br₃, Cl, —Cl₂, —Cl₃, —I, —I₂, —I₃, —OH, alkyl, —O-alkyl, NR′R″, S-alkyl, —SO-alkyl, —SO₂-alkyl, S-aryl, —SO-aryl, —SO₂-aryl, —SO₂—N-aryl, —N—SO₂-arylalkenyl, alkynyl, aryl, heteroaryl, cycloalkyl or heterocyclyl and the aryl or heteroaryl group is optionally substituted with one or more substituents selected from the group consisting of halogen, —OH, alkyl, —O-alkyl, —COOH, —C(O), —C alkyl, —C(O)OC and NR′R″;
R₁ is independently selected from H, alkyl, alkenyl, alkynyl, acyl, aryl, heteroaryl, cycloalkyl, or heterocyclyl, wherein the alkyl, alkenyl, alkynyl, aryl, and acyl group is optionally substituted with one or more groups, independently selected from the group consisting of halogen, —F, —F₂, —F₃, —Br, —Br₂, —Br₃, Cl, —Cl₂, —Cl₃, —I, —I₂, —I₃, —OH, alkyl, —O-alkyl, —O-haloalkyl, NR′R″, S-alkyl, —SO-alkyl, —SO₂-alkyl, S-aryl, —SO-aryl, —SO₂-aryl, —SO₂—N-aryl, —N—SO₂-arylalkenyl, alkynyl, aryl, heteroaryl, cycloalkyl or heterocyclyl and the aryl or heteroaryl group is optionally substituted with one or more substituents selected from the group consisting of halogen, OH, alkyl, —O-alkyl, —COOH, —C(O), —C alkyl, —C(O)OC, NR′R″;
R₂ is selected from H, alkyl, alkyl aryl and aryl;
R′ and R″ are independently selected from the group consisting of H, alkyl, alkenyl, alkynyl or acyl.

The present invention provides a ring annulated cannabidiol analogue compound of Formula I

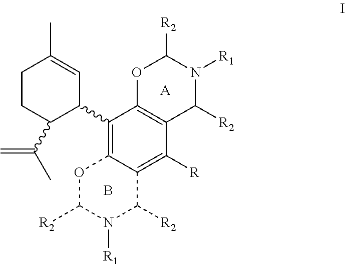

wherein, both A and B rings are simultaneously present or at least one of the ring is present;
R is selected from H, alkyl, alkenyl, alkynyl, acyl, aryl, heteroaryl, cycloalkyl and heterocyclyl, wherein the alkyl, alkenyl, alkynyl or acyl group is optionally substituted with one or more groups, independently selected from the group consisting of halogen, —F, —F₂, —F₃, —Br, —Br₂, —Br₃, Cl, —Cl₂, —Cl₃, —I, —I₂, —I₃, —OH, alkyl, —O-alkyl, NR′R″, S-alkyl, —SO-alkyl, —SO₂-alkyl, S-aryl, —SO-aryl, —SO₂-aryl, —SO₂—N-aryl, —N—SO₂-arylalkenyl, alkynyl, aryl, heteroaryl, cycloalkyl or heterocyclyl and the aryl or heteroaryl group is optionally substituted with one or more substituents selected from the group consisting of halogen, —OH, alkyl, —O— alkyl, —COOH, —C(O), —C alkyl, —C(O)OC and NR′R″;
R₁ is independently selected from H, alkyl, alkenyl, alkynyl, acyl, aryl, heteroaryl, cycloalkyl, or heterocyclyl, wherein the alkyl, alkenyl, alkynyl, aryl, or acyl group is optionally substituted with one or more groups, independently selected from the group consisting of halogen, —F, —F₂, —F₃, —Br, —Br₂, —Br₃, Cl, —Cl₂, —Cl₃, —I, —I₂, —I₃, —OH, alkyl, —O-alkyl, —O-haloalkyl, NR′R″, S-alkyl, —SO-alkyl, —SO₂-alkyl, S-aryl, —SO-aryl, —SO₂-aryl, —SO₂—N-aryl, —N—SO₂-arylalkenyl, alkynyl, aryl, heteroaryl, cycloalkyl or heterocyclyl and the aryl or heteroaryl group is optionally substituted 5 with one or more substituents selected from the group consisting of halogen, OH, alkyl, —O-alkyl, —COOH, —C(O), —C alkyl, —C(O)OC, NR′R″;
R₂ is selected from H, alkyl, alkyl aryl and aryl;
R′ and R″ are independently selected from the group consisting of H, alkyl, alkenyl, alkynyl or acyl.

The process for the synthesis of ring annulated analogues of (N-alkyldihydrol-2H-oxazinyl)-cannabidiol of Formula I

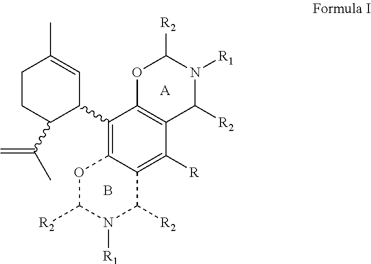

wherein R, R₁ and R₂ are as defined above, comprises of:
coupling of fragment II and fragment III in the presence of aldehyde fragment IV;

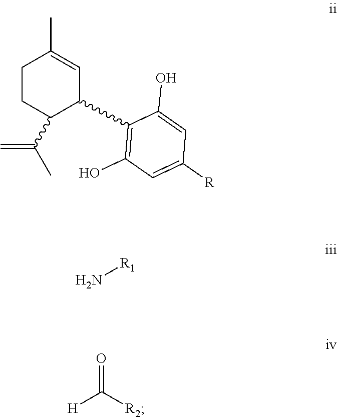

wherein R, R₁ and R₂ in II, III and IV respectively are as defined above; and purifying the obtained compound. The coupling is carried out in presence of a solvent at a temperature in the range of 25-30° C., for a period in the range of 13-16 hours.

There are two stereo-centre in the molecules, wherein [symbol] represents a single bond it may be above the plane or below the plane. The stereo-centre may be R, S as well as mixture of both.

The present invention relates to the synthesis of ring annulated analogues of Formula I by the coupling of fragment II and fragment III in the presence of aldehyde fragment IV in a solvent as provided in Scheme 1.

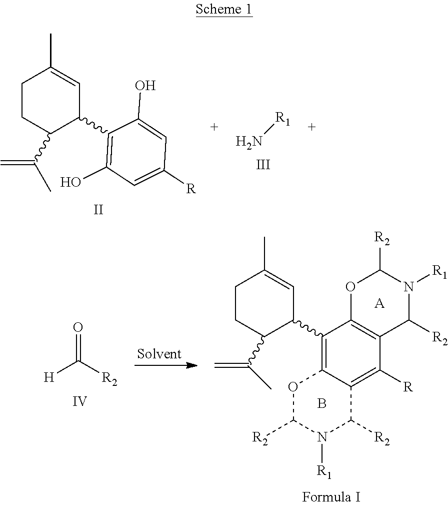

R, R₁ and R₂ in II, III and IV respectively are as defined above. The aldehyde IV is selected from the group consisting of formaldehyde, propionaldehyde, acetaldehydes, propionaldehyde, butyraldehyde, isovaleraldehyde, benzaldehyde, cinnamaldehyde either alone or in combination thereof. The solvent is a single solvent or a mixture of one or more solvents and the solvent is selected from the group consisting of dichloromethane, chloroform, isopropanol, acetone, acetonitrile methanol either alone or in combination thereof.

A preferred embodiment of the present invention relates to the synthesis of a compound of Formula Ia namely (N-methyldihydrol-2H-oxazinyl)-cannabidiol by the coupling of fragment IIa namely cannabidiol and fragment IIIa namely methanamine hydrochloride in the presence of fragment IVa in a solvent.

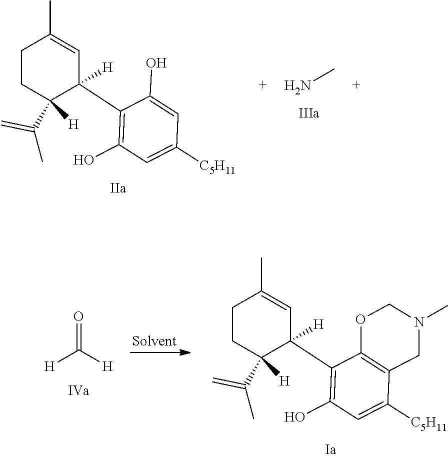

A preferred embodiment of the present invention relates to the synthesis of compound of Formula Ib namely (N-ethyldihydrol-2H-oxazinyl)-cannabidiol by the coupling of fragment IIa namely cannabidiol and fragment IIIb namely ethanamine hydrochloride in the presence of fragment IVa in a solvent.

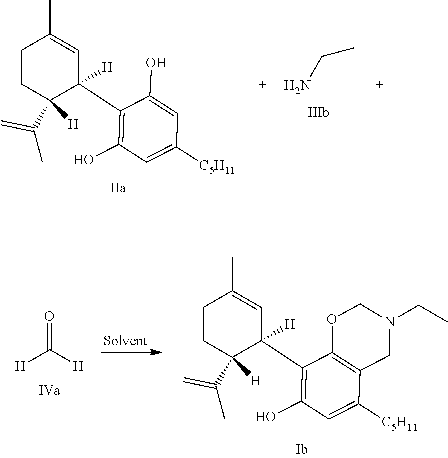

Another preferred embodiment of the present invention relates to the synthesis of a compound of Formula Ic namely (N-propyldihydrol-2H-oxazinyl)-cannabidiol by the coupling of fragment IIa namely cannabidiol and fragment IIIc namely propan-1-amine in the presence of fragment IVa in a solvent.

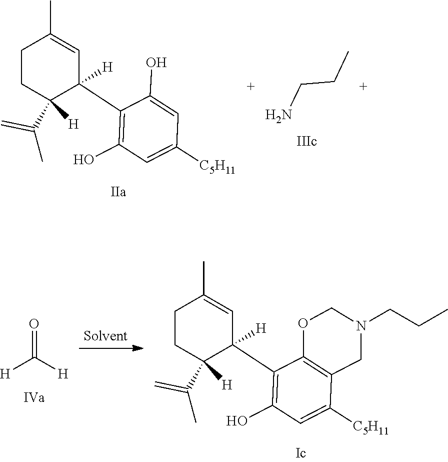

A preferred embodiment of the present invention relates to the synthesis of a compound of Formula Id namely (N-butyldihydrol-2H-oxazinyl)-cannabidiol and Id′ namely bis-{(N-butyldihydrol-2H-oxazinyl)}-cannabidiol by the coupling of fragment IIa namely cannabidiol and fragment IIId namely butan-1-amine in the presence of fragment IVa in a solvent.

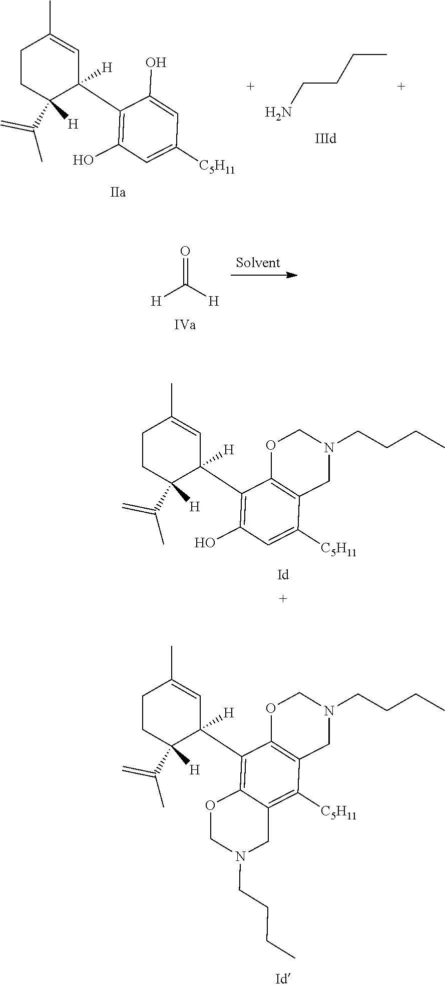

The present invention provides a process for the synthesis of a compound of Formula Ie namely (N-hexyldihydrol-2H-oxazinyl)-cannabidiol by the coupling of fragment IIa namely cannabidiol and fragment IIIe namely hexan-1-amine in the presence of fragment IVa in a solvent.

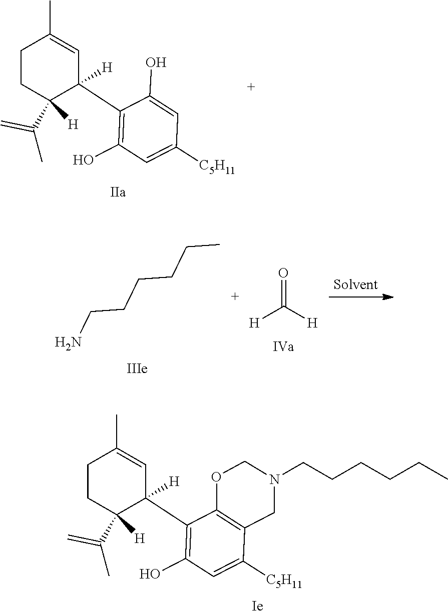

The present invention relates to the synthesis of a compound of Formula If namely (N-morpholinopropyldihydrol-2H-oxazinyl)-cannabidiol by the coupling of fragment IIa namely cannabidiol and fragment IIIf namely 3-morpholinopropan-1-amine in the presence of fragment IVa in a solvent.

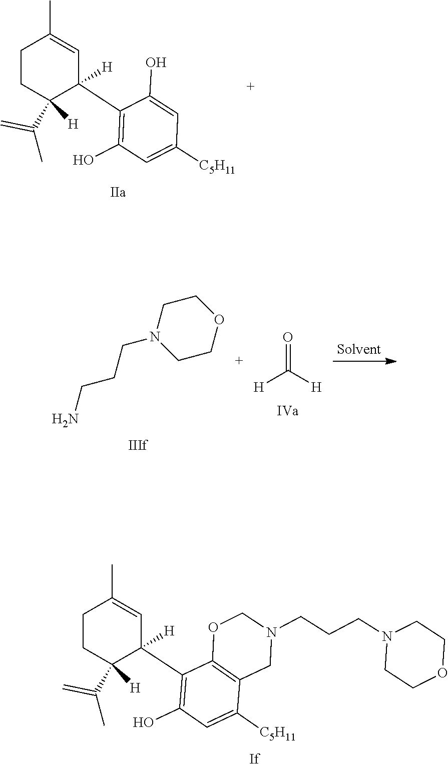

The present invention relates to the synthesis of a compound of Formula Ig namely (N-cyclopropyldihydrol-2H-oxazinyl)-cannabidiol by the coupling of fragment IIa namely cannabidiol and fragment IIIg namely cyclopropanamine in the presence of fragment IVa in a solvent.

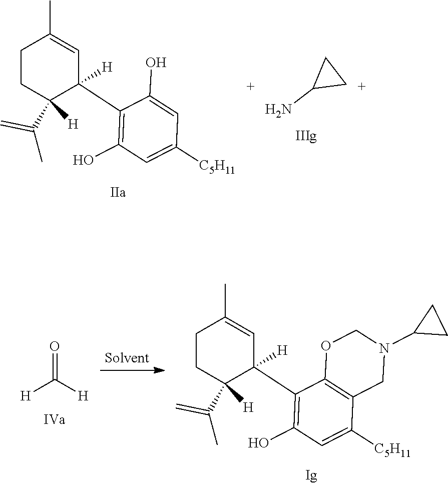

The present invention relates to the synthesis of a compound of Formula Ih namely (N-cyclobutyldihydrol-2H-oxazinyl)-cannabidiol by the coupling of fragment IIa namely cannabidiol and fragment IIIh namely cyclobutanamine in the presence of fragment IVa in a solvent.

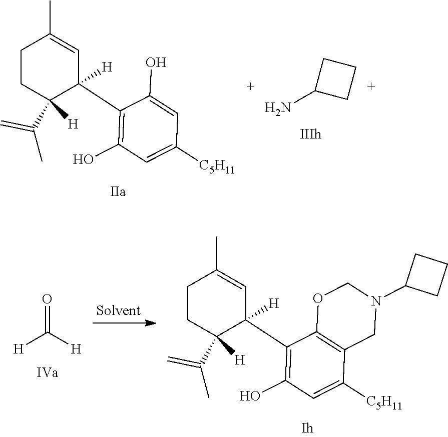

The present invention relates to the synthesis of a compound of Formula II namely (N-cycloheptyldihydrol-2H-oxazinyl)-cannabidiol by the coupling of fragment IIa namely cannabidiol and fragment Iiii namely cycloheptanamine in the presence of fragment IVa in a solvent.

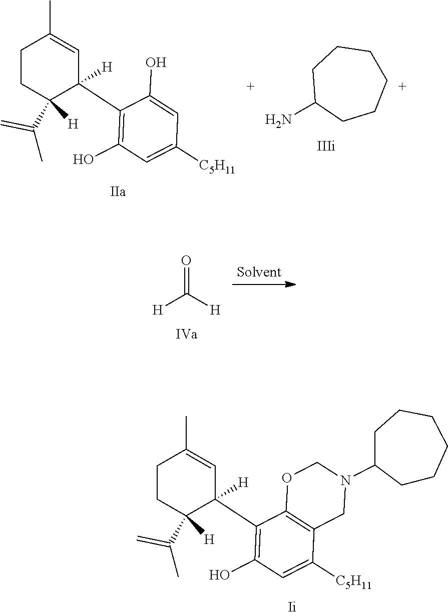

The present invention relates to the synthesis of a compound of Formula Ij namely (N-phenyldihydrol-2H-oxazinyl)-cannabidiol by the coupling of fragment IIa namely cannabidiol and fragment IIIj namely aniline in the presence of fragment IVa in a solvent.

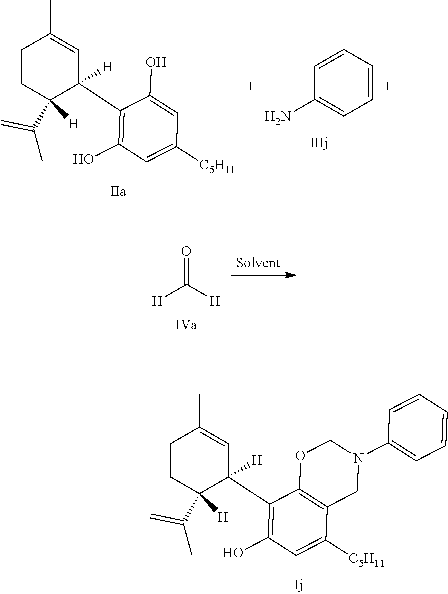

The present invention relates to the synthesis of a compound of Formula Ik namely (N-isopropylphenyldihydrol-2H-oxazinyl)-cannabidiol by the coupling of fragment IIa namely cannabidiol and fragment IIIk namely 4-isopropylaniline in the presence of fragment IVa in a solvent.

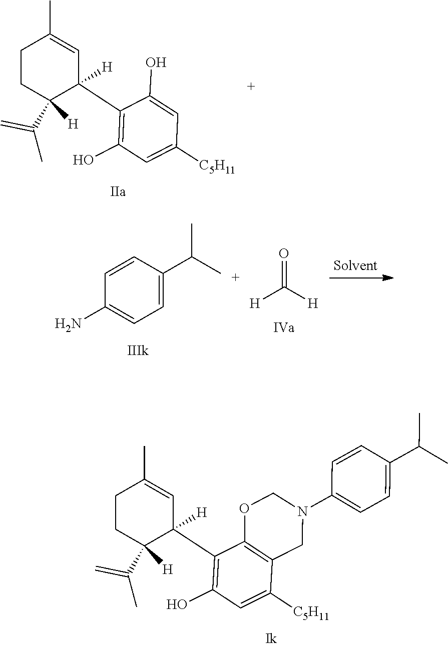

The present invention relates to the synthesis of a compound of Formula Il namely (N-trifluoromethoxyphenyldihydrol-2H-oxazinyl)-cannabidiol by the coupling of fragment IIa namely cannabidiol and fragment IIII namely 4-(trifluoromethoxy)aniline in the presence of fragment IVa in a solvent.

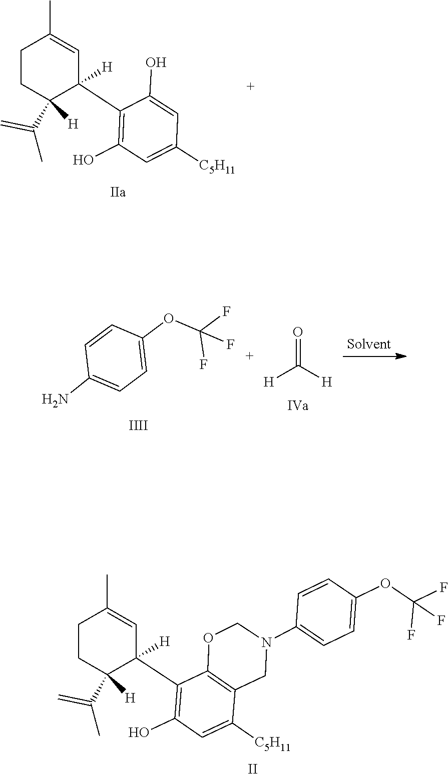

In another embodiment, the present invention relates to the synthesis of a compound of Formula Im namely (N-quinolinedihydrol-2H-oxazinyl)-cannabidiol by the coupling of fragment IIa namely cannabidiol and fragment IIIm namely quinolin-3-amine in the presence of fragment IVa in a solvent.

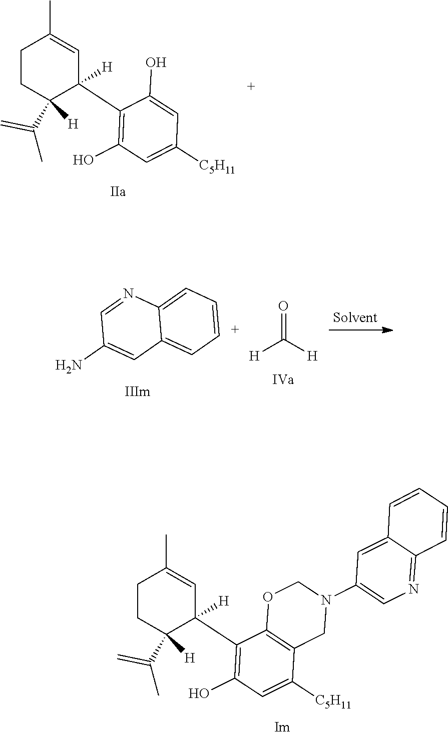

The present invention relates to the synthesis of a compound of Formula In namely (N-benzyldihydrol-2H-oxazinyl)-cannabidiol by the coupling of fragment IIa namely cannabidiol and fragment IIIn namely phenylmethanamine in the presence of fragment IVa in a solvent.

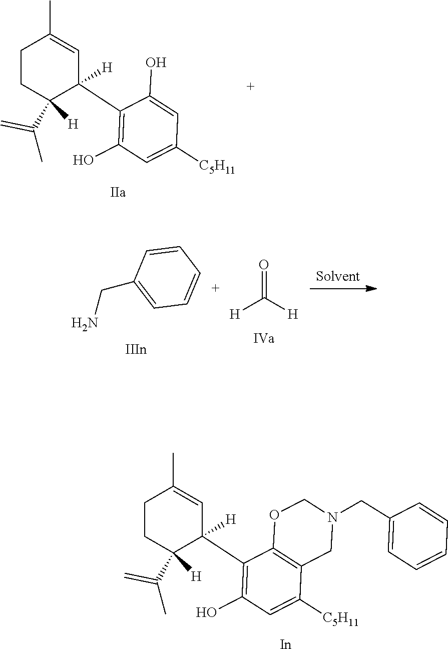

The present invention relates to the synthesis of a compound of Formula Io namely (N-2-fluorophenyldihydrol-2H-oxazinyl)-cannabidiol by the coupling of fragment IIa namely cannabidiol and fragment IIIo namely 2-fluoroaniline in the presence of fragment IVa in a solvent.

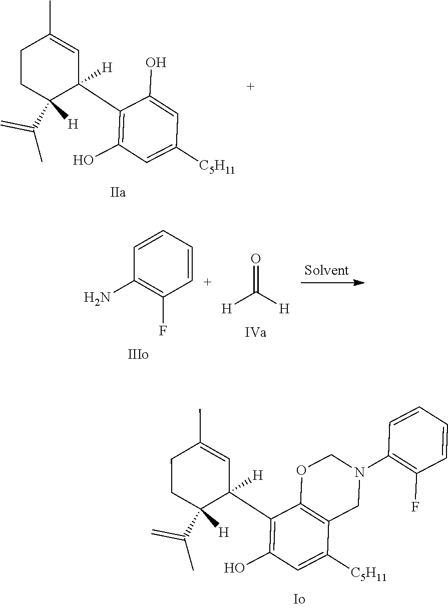

### Material and Method Used in Experiments

All the product mixtures were analyzed by thin layer chromatography. All synthesized derivatives were analyzed by a charring regent such as anisaldehyde solution, dragendroff's solution and ninhydrin's solution. All the reactions were performed under an inert atmosphere wherever required. NMR spectra (¹HNMR, ¹³C, DEPT) were recorded in 400 MHz spectrometer using CDCl₃ and CD₃OD solvent. ES1-MS and HRMS spectra were recorded on LC-MS/MS and HRMS-6540-UHD machines. Optical rotations were measured on a Perkin Elmer polarimeter. Column chromatography was carried out with silica gel (60-120, 100-200 and 230-400 mesh).

### Examples

It should be understood that the disclosed examples are merely exemplary of the invention, which may be embodied in various forms. Therefore, the details disclosed herein are not to be interpreted as limiting but merely as the basis for the claims and as a basis for teaching one skilled in the art how to make or use the invention.

The following examples are given by way of illustration only and therefore should not be construed to limit the scope of the present invention in any manner.

### Example 1: Synthesis of ring annulated analogues of (N-methyldihydrol-2H-oxazinyl)-cannabidiol (Ia)

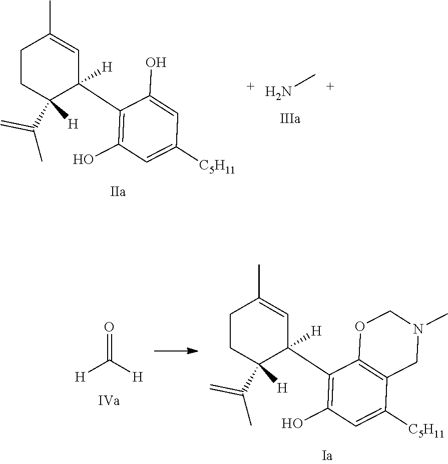

To a solution of cannabidiol (IIa, 200 mg, 0.636 mmol) in methanol formaldehyde solution (IVa, 37%, 128.7 μl) was added followed by methanamine hydrochloride (IIIa, 51.5 mg, 0.763 mmol) to obtain a reaction mixture. The reaction mixture was stirred at room temperature (rt) for 15 hours. The reaction mixture was monitored by thin layer chromatography (TLC) and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-methyldihydrol-2H-oxazinyl)-cannabidiol Ia (CS-58). R_{f}~0.3 (10:90, EtOAc:Hexane), Yield: 80% (190 mg); ¹H NMR (400 MHz, CDCl₃) δ 6.27 (s, 1H), 5.94 (bs, 1H, OH), 5.59 (s, 1H), 4.69 (d, J=9.3 Hz, 1H), 4.57 (d, J=9.3 Hz, 1H), 4.40 (d, J=64.5 Hz, 2H), 3.96-3.86 (m, 2H), 3.75 (d, J=16.0 Hz, 1H), 2.52 (s, 3H), 2.41-2.30 (m, 3H), 2.21-2.04 (m, 2H), 1.78 (s, 5H), 1.67 (s, 3H), 1.55-1.47 (m, 2H), 1.35-1.30 (m, 4H), 0.89 (t, J=6.9 Hz, 3H); ¹³C {¹H}NMR (101 MHz, CDCl₃) δ 153.9, 151.8, 147.3, 139.7, 139.6, 124.4, 114.3, 111.0, 109.2, 108.9, 83.0, 50.2, 46.9, 39.7, 35.1, 31.8, 31.7, 30.4, 29.5, 28.0, 23.7, 22.5, 18.6, 14.0; [α]_{D}²⁰=−83 (c=1.0, MeOH); HRMS (ESI-TOF) m/z: calcd for C₂₄H₃₆NO₂ 370.2746 [M+H]; found 370.2730.

### Example 2: Synthesis of ring annulated analogues of (N-ethyldihydrol-2H-oxazinyl)-cannabidiol (Ib)

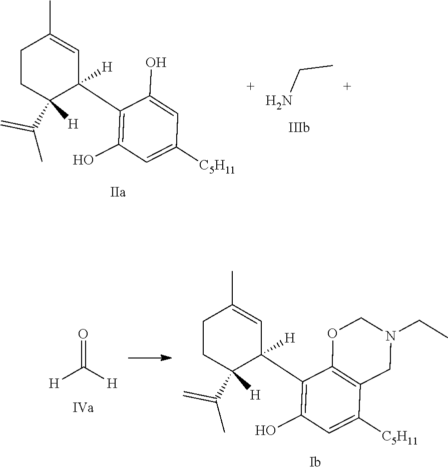

To a solution of cannabidiol (IIa, 200 mg, 0.636 mmol) in methanol formaldehyde solution (IVa, 37%, 128.7 μl) was added followed by ethanamine hydrochloride (IIIb, 62.215 mg, 0.763 mmol) to obtain a reaction mixture. The reaction mixture was stirred at room temperature for 14 hours. The reaction mixture was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-ethyldihydrol-2H-oxazinyl)-cannabidiol Ib (CS-60). R_{f}~0.6 (10:90, EtOAc:Hexane), Yield: 75% (183 mg); ¹H NMR (400 MHz, CDCl₃) δ 6.26 (s, 1H), 5.92 (bs, 1H, OH), 5.58 (s, 1H), 4.77 (d, J=9.6 Hz, 1H), 4.68 (d, J=10.5 Hz, 1H), 4.39 (d, J=61.2 Hz, 2H), 3.95 (d, J=16.2 Hz, 2H), 3.79 (d, J=16.2 Hz, 1H), 2.78-2.64 (m, 2H), 2.44-2.31 (m, 3H), 2.20-2.04 (m, 2H), 1.78 (s, 5H), 1.67 (s, 3H), 1.55-1.47 (m, 2H), 1.33-1.30 (m, 4H), 1.16 (t, J=7.2 Hz, 3H), 0.89 (t, J=6.9 Hz, 3H); ¹³C {¹H}NMR (101 MHz, CDCl₃) δ 153.9, 152.2, 147.3, 139.5, 124.4, 114.3, 111.0, 109.2, 109.1, 80.8, 48.1, 46.9, 45.2, 35.1, 31.8, 31.7, 30.4, 29.6, 28.0, 23.7, 22.5, 18.6, 14.0, 13.3; [α]_{D}²⁰=−83 (c=1.0, MeOH); HRMS (ESI-TOF) m/z: calcd for C₂₅H₃₈NO₂ 384.2903 [M+H]; found 384.2905.

### Example 3: Synthesis of ring annulated analogues of (N-propyldihydrol-2H-oxazinyl)-cannabidiol (Ic)

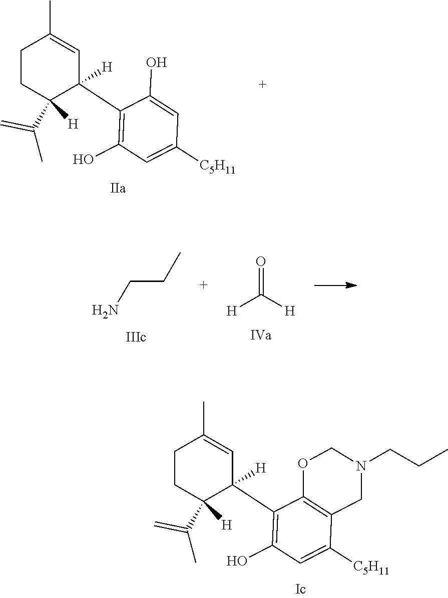

To a solution of cannabidiol (IIa, 200 mg, 0.636 mmol) in methanol formaldehyde solution (IVa, 37%, 128.7 μl) was added followed by propan-1-amine (IIIc, 45 mg, 0.763 mmol) to obtain a reaction mixture. The reaction mixture was stirred at room temperature for 15 hours. The reaction mixture was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-propyldihydrol-2H-oxazinyl)-cannabidiol Ic (CS-56). R_{f}~0.6 (10:90, EtOAc:Hexane), Yield: 78% (197 mg); ¹H NMR (400 MHz, CDCl₃) δ 6.25 (s, 1H), 5.91 (s, 1H), 5.58 (s, 1H), 4.76 (d, J=9.6 Hz, 1H), 4.66 (d, J=8.8 Hz, 1H), 4.47-4.32 (d, J=60 Hz, 2H), 3.94-3.91 (m, 2H), 3.78 (d, J=16.2 Hz, 1H), 2.65-2.53 (m, 2H), 2.42-2.30 (m, 3H), 2.24-2.02 (m, 2H), 1.77 (s, 5H), 1.67 (s, 3H), 1.58-1.49 (m, 4H), 1.33-1.30 (m, 4H), 0.94-0.87 (m, 6H); ¹³C {¹H}NMR (101 MHz, CDCl₃) δ 153.8, 152.2, 147.3, 139.6, 124.4, 114.3, 111.1, 109.3, 109.0, 81.4, 53.2, 48.4, 46.9, 35.1, 31.8, 31.7, 30.4, 29.6, 28.1, 23.7, 22.5, 21.2, 18.6, 14.0, 11.7; [α]_{D}²⁰=−93 (c=1.0, MeOH; HRMS (ESI-TOF) m/z: calcd for C₂₆H₄₀NO₂ 398.3059 [M+H]; found 398.3059.

### Example 4: Synthesis of ring annulated analogues of (N-butyldihydrol-2H-oxazinyl)-cannabidiol (Id) and bis-{(N-butyldihydrol-2H-oxazinyl)}-cannabidiol (Id′)

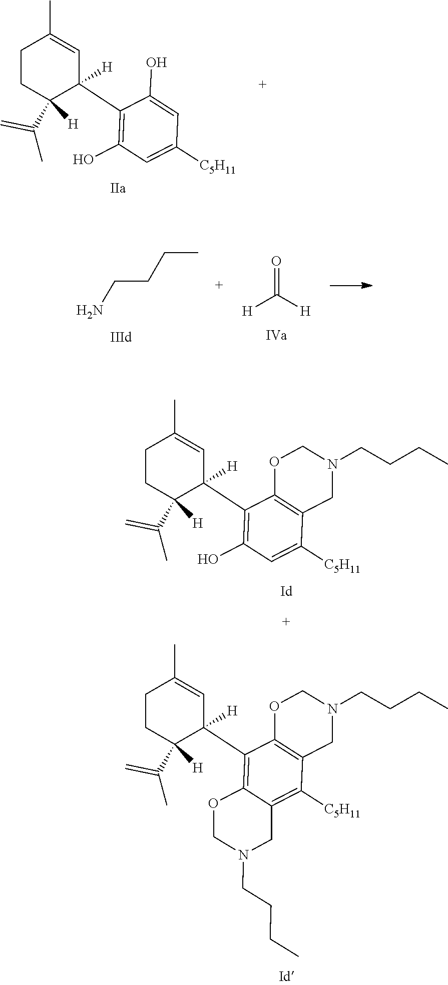

To a solution of cannabidiol (IIa, 200 mg, 0.636 mmol) in methanol formaldehyde solution (IVa, 37%, 210 μl) was added followed by butan-1-amine (IIId, 139.2 mg, 1.908 mmol) to obtain a reaction mixture. The reaction mixture was stirred at room temperature for 15 hours. The reaction mixture was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-butyldihydrol-2H-oxazinyl)-cannabidiol Id (CS-20) and bis-{(N-butyldihydrol-2H-oxazinyl)}-cannabidiol Id′ (CS-71). R_{f}~0.7 (10:90, EtOAc:Hexane), Yield: 30.2% (80 mg); ¹H NMR (400 MHz, CDCl₃) δ 6.18 (s, 1H), 5.83 (s, 1H), 5.51 (s, 1H), 4.69 (d, J=9.6 Hz, 1H), 4.59 (d, J=9.6 Hz, 1H), 4.32 (d, J=59.9 Hz, 2H), 3.95-3.80 (m, 2H), 3.71 (d, J=16.2 Hz, 1H), 2.61-2.52 (m, 2H), 2.33-2.24 (m, 3H), 2.13-1.97 (m, 2H), 1.70 (s, 5H), 1.60 (s, 3H), 1.47-1.40 (m, 4H), 1.31-1.29 (m, 2H), 1.27-1.24 (m, 4H), 0.88-0.80 (m, 6H); ¹³C {¹H}NMR (101 MHz, CDCl₃) δ 153.9, 152.3, 147.3, 139.6, 124.4, 114.3, 111.1, 109.3, 109.0, 81.4, 51.0, 48.4, 46.9, 35.1, 31.8, 31.7, 30.4, 30.2, 29.6, 28.1, 23.7, 22.6, 20.5, 18.6, 14.06; [α]_{D}²⁰=−122 (c=1.0, MeOH; HRMS (ESI-TOF) m/z: calcd for C₂₇H₄₂NO₂ 412.3216 [M+H]; found 412.3192. R_{f}~0.5, (10:90, EtOAc:Hexane), Yield: 52% (168 mg); ¹H NMR (400 MHz, CDCl₃) δ 5.23 (s, 1H), 4.72 (s, 4H), 4.45 (d, J=6.6 Hz, 2H), 3.91 (d, J=12.3 Hz, 5H), 2.95-2.88 (m, 1H), 2.72-2.60 (m, 4H), 2.27-1.94 (m, 4H), 1.79-1.69 (m, 2H), 1.63 (d, J=9.6 Hz, 6H), 1.57-1.49 (m, 4H), 1.40-1.31 (m, 10H), 0.93 (t, J=7.3 Hz, 9H); ¹³C {¹H}NMR (101 MHz, CDCl₃) δ 149.6, 135.7, 130.9, 125.9, 116.4, 109.8, 109.6, 81.1, 50.9, 48.5, 44.9, 35.6, 32.3, 30.7, 30.3, 29.7, 29.1, 27.5, 23.4, 22.5, 20.5, 18.9, 14.0; [α]_{D}²⁰=−127 (c=1.0, MeOH); HRMS (ESI-TOF) m/z: calcd for C₃₃H₅₃N₂O₂ 509.4107 [M+H]; found 509.4106.

### Example 5: Synthesis of ring annulated analogues of (N-hexyldihydrol-2H-oxazinyl)-cannabidiol (Ie)

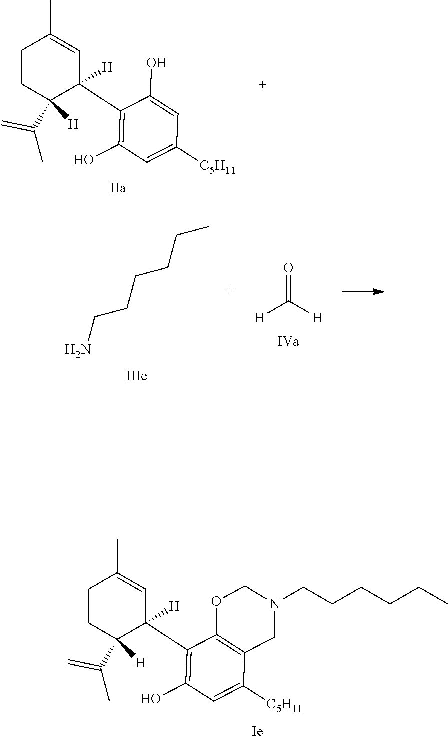

To a solution of cannabidiol (IIa, 200 mg, 0.636 mmol) in methanol formaldehyde solution (IVa, 37%, 128.7 μl) was added followed by hexan-1-amine (IIIe, 105 mg, 0.763 mmol) to obtain a reaction mixture. The reaction mixture was stirred at room temperature for 13 hours. The reaction mixture was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-hexyldihydrol-2H-oxazinyl)-cannabidiol he (CS-61). R_{f}~0.7 (10:90, EtOAc:Hexane), Yield: 70% (198 mg); ¹H NMR (400 MHz, CDCl₃) δ 6.25 (s, 1H), 5.92 (s, 1H), 5.58 (s, 1H), 4.76 (d, J=9.6 Hz, 1H), 4.66 (d, J=9.3 Hz, 1H), 4.39 (d, J=61.3 Hz, 2H), 3.97-3.90 (m, 2H), 3.78 (d, J=16.2 Hz, 1H), 2.70-2.56 (m, 2H), 2.41-2.31 (m, 3H), 2.24-2.05 (m, 2H), 1.78 (s, 5H), 1.67 (s, 3H), 1.54-1.49 (m, 4H), 1.36-1.28 (m, 10H), 0.90-0.87 (m, 6H); ¹³C {¹H}NMR (101 MHz, CDCl₃) δ 153.95, 152.32, 147.34, 139.5, 139.4, 124.5, 114.3, 111.1, 109.2, 109.1, 81.4, 51.4, 48.4, 46.9, 35.2, 31.9, 31.8, 31.7, 30.4, 29.7, 28.1, 27.1, 23.7, 22.7, 22.6, 18.7, 14.1, 14.0; [α]_{D}²⁰=93 (c=1.0, MeOH); HRMS (ESI-TOF) m/z: calcd for C₂₉H₄₆NO₂ 440.3529 [M+H]; found 440.3530.

### Example 6: Synthesis of ring annulated analogues of (N-morpholinopropyldihydrol-2H-oxazinyl)-cannabidiol (If)

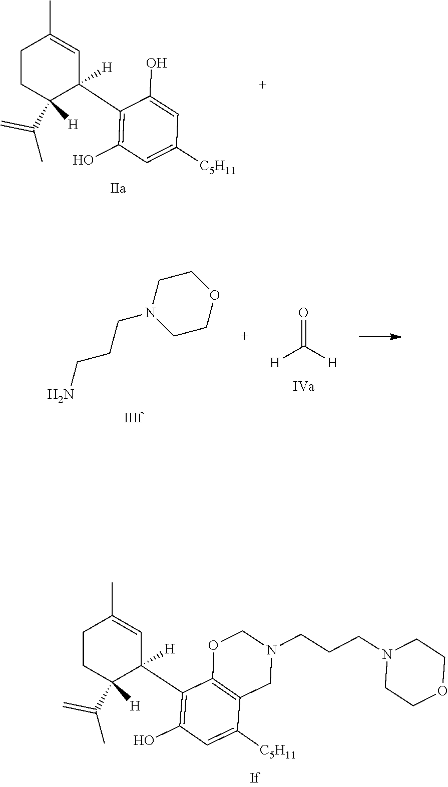

To a solution of cannabidiol (IIa, 200 mg, 0.636 mmol) in methanol formaldehyde solution (IVa, 37%, 128.741) was added followed by 3-morpholinopropan-1-amine (IIIf, 109.8 mg, 0.763 mmol) to obtain a reaction mixture. The reaction mixture was stirred at room temperature for 15 hours. The reaction mixture was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-morpholinopropyldihydrol-2H-oxazinyl)-cannabidiol If (CS-62). R_{f}~0.5 (80:20, EtOAc:Hexane), Yield: 90% (277 mg); ¹H NMR (400 MHz, CDCl₃) δ 6.26 (s, 1H), 5.93 (bs, 1H, OH), 5.58 (s, 1H), 4.75 (d, J=9.7 Hz, 1H), 4.64 (d, J=9.5 Hz, 1H), 4.39 (d, J=60.2 Hz, 2H), 3.94-3.70 (m, 7H), 2.74-2.63 (m, 2H), 2.42-2.31 (m, 7H), 2.27-2.02 (m, 4H), 1.78-1.70 (m, 7H, 1.67 (s, 3H), 1.56-1.44 (m, 2H), 1.34-1.24 (m, 4H), 0.89 (t, J=6.9 Hz, 3H); ¹³C {¹H}NMR (101 MHz, CDCl₃) δ 153.9, 152.2, 147.3, 139.7, 139.6, 124.4, 114.3, 111.0, 109.2, 109.1, 81.5, 66.9, 56.9, 53.8, 49.3, 48.4, 46.8, 35.1, 31.8, 31.7, 30.4, 29.6, 28.0, 25.1, 23.7, 22.5, 18.7, 14.0; [α]_{D}²⁰=−77 (c=1.0, MeOH); HRMS (ESI-TOF) m/z: calcd for C₃₀H₄₇N₂O₃ 483.3587 [M+H]; found 483.3582.

### Example 7: Synthesis of ring annulated analogues of (N-cyclopropyldihydrol-2H-oxazinyl)-cannabidiol (Ig)

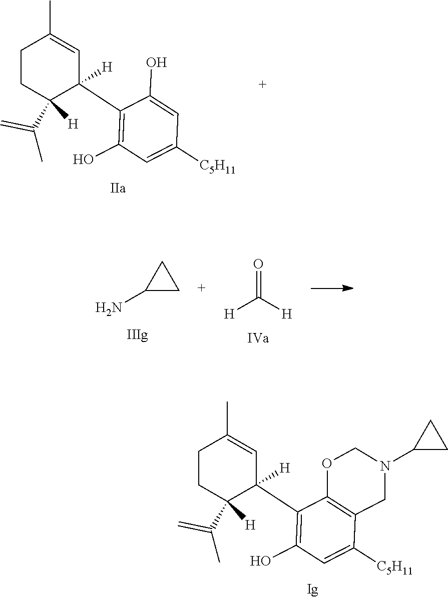

To a solution of cannabidiol (IIa, 200 mg, 0.636 mmol) in methanol formaldehyde solution (IVa, 37%, 128.7 μl) was added followed by cyclopropanamine (IIIg, 43.5 mg, 0.763 mmol) to obtain a reaction mixture. The reaction mixture was stirred at room temperature for 16 hours. The reaction mixture was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-cyclopropyldihydrol-2H-oxazinyl)-cannabidiol Ig (CS-57). R_{f}~0.7 (10:90, EtOAc:Hexane), Yield: 70% (178 mg); ¹H NMR (400 MHz, CDCl₃) δ 6.28 (s, 1H), 5.94 (s, 1H), 5.60 (s, 1H), 4.78 (d, J=9.3 Hz, 1H), 4.66 (d, J=9.2 Hz, 1H), 4.37 (d, J=48.8 Hz, 2H), 3.99-3.88 (m, 3H), 2.39-2.31 (m, 4H), 2.21-2.05 (m, 2H), 1.79-1.76 (m, 5H), 1.68 (s, 3H), 1.56-1.51 (m, 2H), 1.34-1.31 (m, 4H), 0.89 (t, J=7.0 Hz, 3H), 0.54-0.51 (m, 4H); ¹³C {¹H}NMR (101 MHz, CDCl₃) δ 153.9, 152.3, 147.5, 139.7, 139.5, 124.4, 114.4, 111.0, 109.6, 109.1, 81.7, 49.0, 47.0, 35.2, 32.7, 31.8, 31.7, 30.4, 29.5, 28.0, 23.8, 22.6, 18.6, 14.1, 7.0, 6.7; [α]_{D}²⁰=−92 (c=1.0, MeOH); HRMS (ESI-TOF) m/z: calcd for C₂₆H₃₈NO₂ 396.2903 [M+H]; found 396.2904.

### Example 8: Synthesis of ring annulated analogues of (N-cyclobutyldihydrol-2H-oxazinyl)-cannabidiol (Ih)

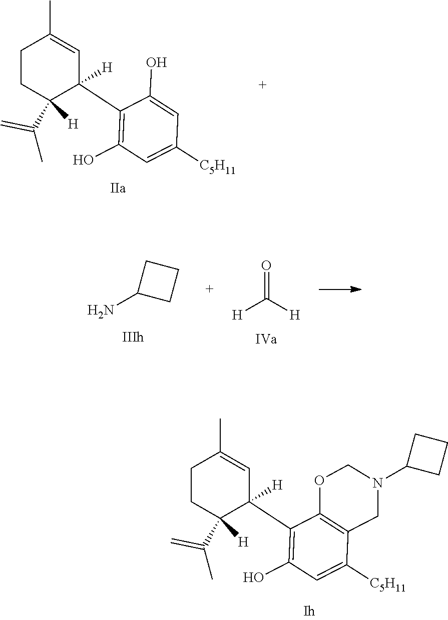

To a solution of cannabidiol (IIa, 200 mg, 0.636 mmol) in methanol formaldehyde solution (IVa, 37%, 128.7 μl) was added followed by cyclobutanamine (IIIh, 54.2 mg, 0.763 mmol) to obtain a reaction mixture. The reaction mixture was stirred at room temperature for 15 hours. The reaction mixture was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-cyclobutyldihydrol-2H-oxazinyl)-cannabidiol Ih, (CS-59). R_{f}~0.7 (10:90, EtOAc:Hexane), Yield: 73% (190 mg); ¹H NMR (400 MHz, CDCl₃) δ 6.18 (s, 1H), 5.84 (s, 1H), 5.50 (s, 1H), 4.63 (d, J=9.6 Hz, 1H), 4.53 (d, J=9.6 Hz, 1H), 4.35 (d, J=69.8 Hz, 2H), 3.85-3.74 (m, 2H), 3.64 (d, J=16.2 Hz, 1H), 3.41-3.27 (m, 1H), 2.36-2.23 (m, 3H), 2.18-1.97 (m, 4H), 1.87-1.76 (m, 2H), 1.75-1.64 (m, 6H), 1.60 (s, 3H), 1.57-1.51 (m, 1H), 1.49-1.39 (m, 2H), 1.30-1.21 (m, 4H), 0.82 (t, J=6.9 Hz, 3H); ¹³C {¹H}NMR (101 MHz, CDCl₃) δ 153.9, 152.5, 147.4, 139.5, 124.5, 114.3, 111.0, 109.2, 109.1, 78.1, 53.1, 46.9, 44.3, 35.1, 31.8, 31.7, 30.4, 29.6, 28.1, 27.9, 27.6, 23.7, 22.6, 18.7, 14.1, 14.0; [α]_{D}²⁰=−93 (c=1.0, MeOH); HRMS (ESI-TOF) m/z: calcd for C₂₇H₄₀NO₂ 410.3059 [M+H]; found 410.3049.

### Example 9: Synthesis of ring annulated analogues of (N-cycloheptyldihydrol-2H-oxazinyl)-cannabidiol (Ii)

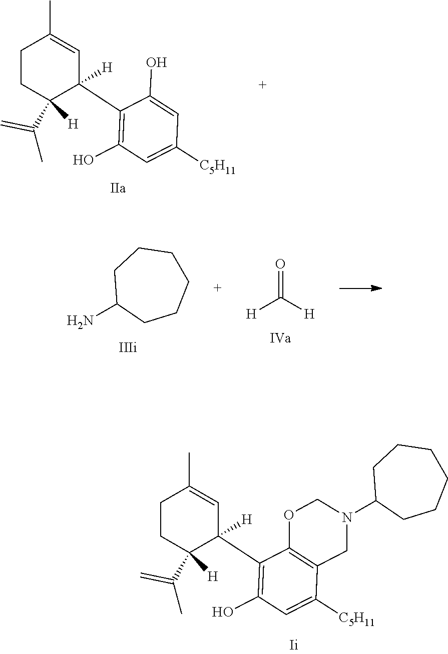

To a solution of cannabidiol (IIa, 200 mg, 0.636 mmol) in methanol formaldehyde solution (IVa, 37%, 128.7 μl) was added followed by cycloheptanamine (IIIi, 86.2 mg, 0.763 mmol) to obtain a reaction mixture. The reaction mixture was stirred at room temperature for 15 hours. The reaction mixture 5 was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-cycloheptyldihydrol-2H-oxazinyl)-cannabidiol Ii (CS-68). R_{f}~0.8 (10:90, EtOAc:Hexane), Yield: 75% (216 mg); ¹H NMR (400 MHz, CDCl₃) δ 6.24 (s, 1H), 5.87 (s, 1H), 5.55 (s, 1H), 4.83-4.74 (m, 2H), 4.40 (d, J=55.2 Hz, 2H), 3.97-3.85 (m, 3H), 2.85-2.81 (m, 1H), 2.43-2.32 (m, 3H), 2.22-2.05 (m, 2H), 1.95-1.81 (m, 3H), 1.80-1.75 (m, 5H), 1.66 (s, 3H), 1.57-1.48 (m, 8H), 1.45-1.37 (m, 3H), 1.34-1.31 (m, 4H), 0.89 (d, J=6.6 Hz, 3H); ¹³C {¹H}NMR (101 MHz, CDCl₃) δ 153.7, 153.2, 147.3, 139.1, 138.9, 124.6, 114.5, 111.0, 110.9, 109.0, 79.9, 61.4, 46.6, 45.5, 35.1, 32.6, 31.8, 31.7, 30.3, 29.5, 28.1, 27.9, 24.8, 24.7, 23.7, 22.6, 18.8, 14.0; [α]_{D}²⁰=−68 (c=1.0, MeOH); HRMS (ESI-TOF) m/z: calcd for C₃₀H₄₆NO₂ 452.3529 [M+H]; found 452.3521.

### Example 10: Synthesis of ring annulated analogues of (N-phenyldihydrol-2H-oxazinyl)-cannabidiol (Ij)

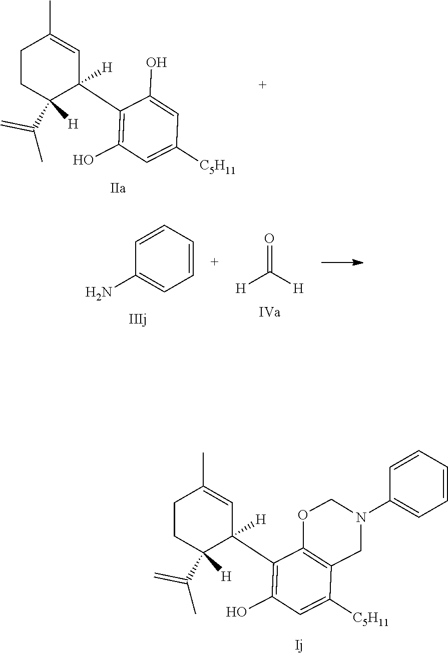

To a solution of cannabidiol (IIa, 200 mg, 0.636 mmol) in methanol formaldehyde solution (IVa, 37%, 128.7 μl) was added followed by aniline (IIIj, 70.9 mg, 0.763 mmol) to obtain a reaction mixture. The reaction mixture was stirred at room temperature for 15 hours. The reaction mixture was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-phenyldihydrol-2H-oxazinyl)-cannabidiol Ij (CS-65). R_{f}~0.7 (10:90, EtOAc:Hexane), Yield: 75% (207 mg); ¹H NMR (400 MHz, CDCl₃) δ 7.18 (t, J=7.9 Hz, 2H), 6.98 (d, J=8.0 Hz, 2H), 6.85 (t, J=7.3 Hz, 1H), 6.22 (s, 1H), 5.85 (s, 1H), 5.48 (s, 1H), 5.17 (d, J=10.1 Hz, 1H), 5.06 (d, J=10.1 Hz, 1H), 4.41 (s, 2H), 4.21 (d, J=25.9 Hz, 2H), 3.83 (d, J=8.8 Hz, 1H), 2.37-2.26 (m, 3H), 2.12-1.96 (m, 2H), 1.69-1.65 (s, 5H), 1.49 (s, 3H), 1.29-1.26 (m, 2H), 1.19 (s, 4H), 0.83 (t, J=6.9 Hz, 3H); ¹³C {¹H}NMR (101 MHz, CDCl₃) δ 154.1, 152.7, 148.9, 146.8, 139.7, 138.8, 129.1, 124.3, 121.2, 118.4, 114.8, 111.2, 110.1, 109.5, 79.1, 48.2, 46.7, 35.3, 31.9, 31.8, 30.4, 29.5, 27.9, 23.7, 22.6, 18.7, 14.1; [α]_{D}²⁰=−109 (c=1.0, MeOH); MS: (ESI+): m/z calcd for C₂₉H₃₈NO₂ 432.29; found 432.35.

### Example 11: Synthesis of ring annulated analogues of (N-isopropylphenyldihydrol-2H-oxazinyl)-cannabidiol (Ik)

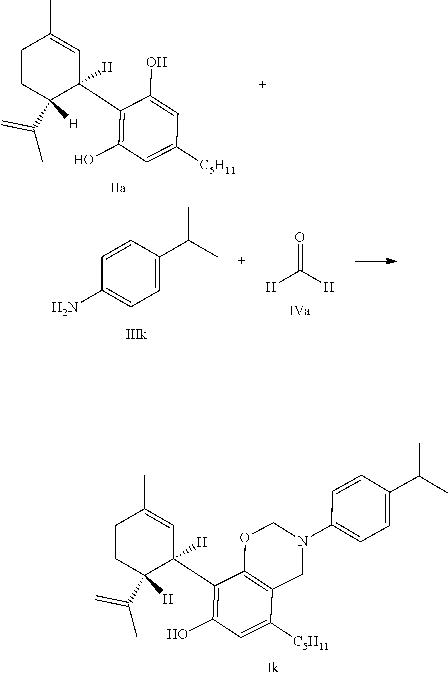

To a solution of cannabidiol (IIa, 200 mg, 0.636 mmol) in methanol formaldehyde solution (IVa, 37%, 128.7 μl) was added followed by 4-isopropylaniline (I₁₁k, 103 mg, 0.764 mmol) to obtain a reaction mixture. The reaction mixture was stirred at room temperature for 15 hours. The reaction mixture was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-isopropylphenyldihydrol-2H-oxazinyl)-cannabidiol Ik (CS-55). R_{f}~0.2 (10:90, EtOAc:Hexane), Yield: 70% (212 mg); ¹H NMR (400 MHz, CDCl₃) δ 7.11 (d, J=6.7 Hz, 2H), 6.98 (d, J=8.6 Hz, 2H), 6.28 (s, 1H), 5.93 (s, 1H), 5.56 (s, 1H), 5.17 (dd, J=41.5, 10.1 Hz, 2H), 4.45 (s, 2H), 4.28 (d, J=21.9 Hz, 2H), 3.90 (d, J=8.7 Hz, 1H), 2.87-2.80 (m, 1H), 2.46-2.30 (m, 3H), 2.26-2.03 (m, 2H), 1.76-1.73 (m, 5H), 1.59 (s, 2H), 1.55 (s, 3H), 1.39-1.32 (m, 4H), 1.22 (d, J=6.9 Hz, 6H), 0.91 (t, J=7.0 Hz, 3H); ¹³C {¹H}NMR (101 MHz, CDCl₃) δ 154.1, 152.8, 146.9, 146.8, 141.9, 139.7, 138.8, 126.9, 124.4, 118.8, 114.8, 111.2, 110.2, 109.4, 79.6, 48.3, 46.7, 35.3, 33.3, 31.8, 31.7, 30.4, 29.4, 28.0, 24.2, 24.1, 23.7, 22.6, 18.7, 14.1; [α]_{D}²⁰=−81 (c=1.0, MeOH); HRMS (ESI-TOF) m/z: calcd for C₃₂H₄₄NO₂ 474.3372 [M+H]; found 474.3372.4

### Example 12: Synthesis of ring annulated analogues of (N-trifluoromethoxyphenyldihydrol-2H-oxazinyl)-cannabidiol (Ii)

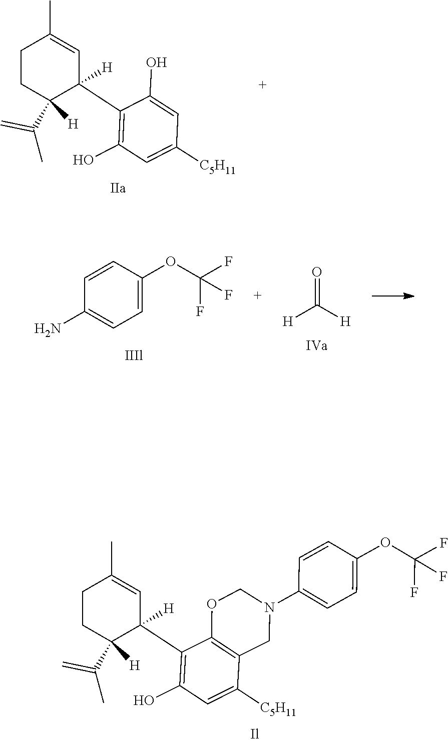

To a solution of cannabidiol (IIa, 200 mg, 0.636 mmol) in methanol formaldehyde solution (IVa, 37%, 128.7 μl) was added followed by 4-(trifluoromethoxy)aniline (IIII, 135.1 mg, 0.763 mmol) to obtain a reaction mixture. The reaction mixture was stirred at room temperature for 15 hours. The reaction mixture was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-trifluoromethoxyphenyldihydrol-2H-oxazinyl)-cannabidiol Il (CS-66). R_{f}~0.7 (10:90, EtOAc:Hexane), Yield: 80% (263 mg); ¹H NMR (400 MHz, CDCl₃) δ 7.12-7.02 (m, 4H), 6.32 (s, 1H), 5.95 (s, 1H), 5.56 (s, 1H), 5.22-5.12 (m, 2H), 4.48-4.43 (m, 2H), 4.27 (d, J=15.8 Hz, 2H), 3.90 (d, J=8.6 Hz, 1H), 2.44-2.32 (m, 3H), 2.24-2.04 (m, 2H), 1.81-1.73 (m, 5H), 1.60-1.53 (m, 5H), 1.39-1.33 (m, 4H), 0.92 (t, J=7.0 Hz, 3H); ¹³C {¹H}NMR (101 MHz, CDCl₃) δ 154.3, 152.6, 147.8, 146.9, 143.3, 139.9, 138.9, 129.6 (q, J=257.5 Hz), 124.2, 121.9, 119.5, 114.9, 111.2, 109.8, 109.7, 79.2, 48.4, 46.7, 35.2, 31.8, 31.7, 30.3, 29.4, 28.0, 23.7, 22.6, 18.6, 14.0; [α]_{D}²⁰=−80 (c=1.0, MeOH); MS: (ESI+): m/z calcd for 5 C₃₀H₃₇F₃NO₃ 516.27; found 516.30.

### Example 13: Synthesis of ring annulated analogues of (N-quinolinedihydrol-2H-oxazinyl)-cannabidiol (Im)

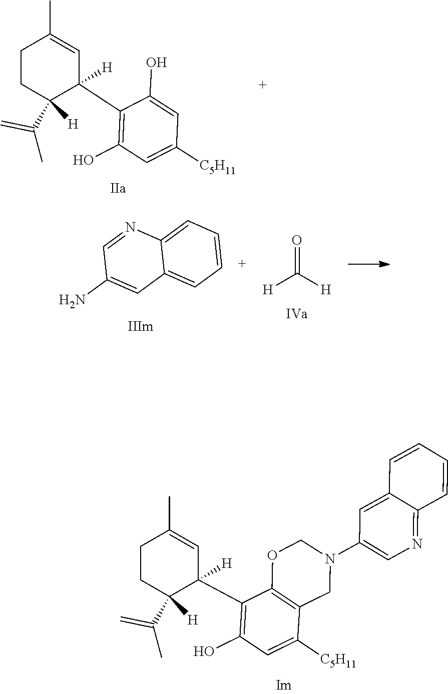

To a solution of cannabidiol (IIa, 200 mg, 0.636 mmol) in methanol formaldehyde solution (IVa, 37%, 128.7 μl) was added followed by quinolin-3-amine (IIIm, 110 mg, 0.764 mmol) to obtain a reaction mixture. The reaction mixture was stirred at room temperature for 15 hours. The reaction mixture was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-quinolinedihydrol-2H-oxazinyl)-cannabidiol) Im (CS-63). R_{f}-0.2 (10:90, EtOAc:Hexane), Yield: 65% (200 mg); ¹H NMR (400 MHz, CDCl₃) δ 8.87 (d, J=2.8 Hz, 1H), 8.01 (d, J=8.4 Hz, 1H), 7.65 (dd, J=8.1, 1.2 Hz, 1H), 7.58-7.51 (m, 2H), 7.50-7.43 (m, 1H), 6.33 (s, 1H), 6.00 (s, 1H), 5.54 (s, 1H), 5.35-5.26 (m, 2H), 4.61 (s, 2H), 4.16 (d, J=22.6 Hz, 2H), 3.91 (d, J 10.3 Hz, 1H), 2.49-2.30 (m, 3H), 2.23-2.03 (m, 2H), 1.79-1.70 (m, 5H), 1.60-1.55 (m, 2H), 1.51 (s, 3H), 1.39-1.35 (m, 4H), 0.91 (t, J=7.0 Hz, 3H); ¹³C {¹H}NMR (101 MHz, CDCl₃) δ 154.4, 152.5, 146.9, 145.9, 143.8, 142.3, 139.9, 138.9, 128.9, 128.5, 127.1, 127.0, 126.8, 124.1, 120.4, 115.0, 111.2, 110.0, 109.3, 78.9, 48.3, 46.6, 35.3, 31.8, 31.7, 30.4, 29.5, 28.0, 23.7, 22.6, 18.7, 14.1; [α]_{D}²⁰=48 (c=1.0, MeOH); HRMS (ESI-TOF) m/z: calcd for C₃₂H₃₉N₂O₂ 483.3012 [M+H]; found 483.3012.

### Example 14: Synthesis of ring annulated analogues of (N-benzyldihydrol-2H-oxazinyl)-cannabidiol (In)

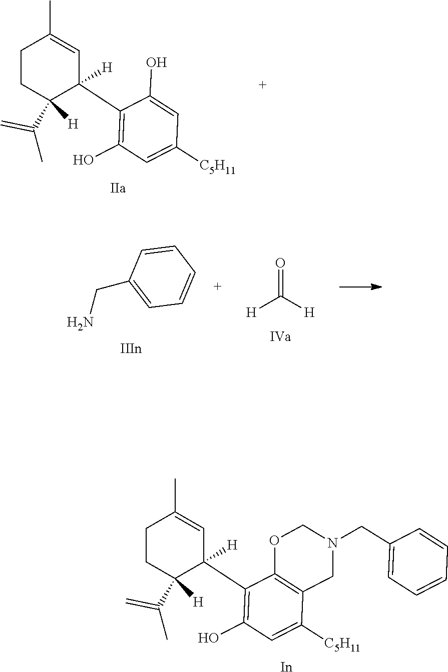

To a solution of cannabidiol (IIa, 200 mg, 0.636 mmol) in methanol formaldehyde solution (IVa, 37%, 128.7 μl) was added followed by phenylmethanamine (IIIn, 81.6 mg, 0.763 mmol) to obtain a reaction mixture. The reaction mixture was stirred at room temperature for 15 hours. The reaction mixture was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-benzyldihydrol-2H-oxazinyl)-cannabidiol In (CS-67). R_{f}~0.7 (10:90, EtOAc:Hexane), Yield: 90% (255 mg); ¹H NMR (400 MHz, CDCl₃) δ 7.30-7.17 (m, 5H), 6.20 (s, 1H), 5.86 (s, 1H), 5.53 (s, 1H), 4.72 (d, J=9.6 Hz, 1H), 4.61 (d, J=9.5 Hz, 1H), 4.40 (d, J=71.6 Hz, 2H), 3.90 (d, J=8.7 Hz, 1H), 3.84-3.65 (m, 4H), 2.36-2.03 (m, 5H), 1.73-1.68 (m, 5H), 1.62 (s, 3H), 1.40-1.33 (m, 2H), 1.19-1.17 (m, 4H), 0.78 (t, J=6.9 Hz, 3H); ¹³C {¹H}NMR (101 MHz, CDCl₃) δ 154.0, 152.2, 147.5, 139.8, 139.6, 138.6, 129.0, 128.4, 127.3, 124.5, 114.4, 111.1, 109.3, 109.0, 81.6, 55.4, 47.6, 46.9, 35.3, 31.8, 31.7, 30.5, 29.6, 28.1, 23.8, 22.6, 18.8, 14.0; [α]_{D}²⁰=−106 (c=1.0, MeOH); HRMS (ESI-TOF) m/z: calcd for C₃₀H₄₀NO₂ 446.3059 [M+H]; found 446.3051.

### Example 15: Synthesis of ring annulated analogues of (N-2-fluorophenyldihydrol-2H-oxazinyl)-cannabidiol (Io)

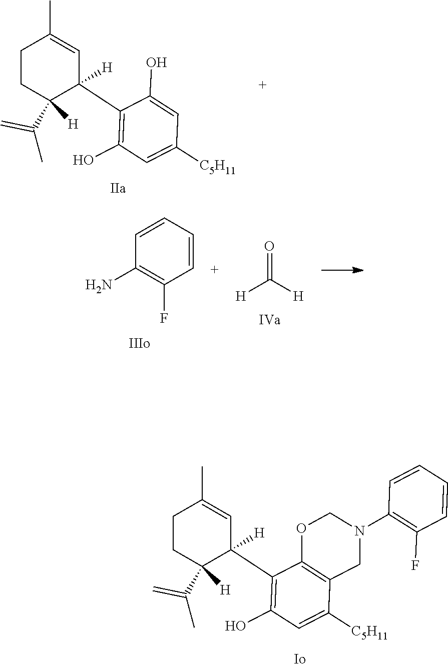

To a solution of cannabidiol (IIa, 200 mg, 0.636 mmol) in methanol formaldehyde solution (IVa, 37%, 128.7 μl) was added followed by 2-fluoroaniline (IIIo, 84.8 mg, 0.763 mmol) to obtain a reaction mixture. The reaction mixture was stirred at room temperature for 15 hours. The reaction mixture was monitored by TLC and observed for consumption of reactant. After the completion of the reaction, the reaction mixture was extracted with ethyl acetate (2×200 mL volume) and water. The organic layer was collected and concentrated in vacuo on rota evaporator. The compounds were purified through column chromatography and eluted with ethyl acetate and hexane to get the (N-2-fluorophenyldihydrol-2H-oxazinyl)-cannabidiol Io (CS-64). R_{f}~0.7 (10:90, EtOAc:Hexane), Yield: 65% (187 mg); ¹H NMR (400 MHz, CDCl₃) δ 7.07-6.96 (m, 4H), 6.31 (s, 1H), 5.94 (s, 1H), 5.56 (s, 1H), 5.18-5.08 (m, 2H), 4.45-4.28 (m, 4H), 3.91 (d, J=8.7 Hz, 1H), 2.44-2.34 (m, 3H), 2.21-2.05 (m, 2H), 1.77-1.73 (m, 5H), 1.56-1.50 (m, 5H), 1.36-1.31 (m, 4H), 0.89 (t, J=7.0 Hz, 3H); ¹³C {¹H}NMR (101 MHz, CDCl₃) δ 155.7 (d, J=245.4), 154.2, 152.4, 146.9, 139.8, 138.9, 137.4, 124.3, 123.6, 121.8, 116.1, 115.9, 114.9, 111.3, 110.0, 109.6, 79.6, 48.5, 46.7, 35.2, 31.79, 31.70, 30.3, 29.4, 28.0, 23.7, 22.5, 18.6, 14.0; [α]_{D}²⁰=−53 (c=1.0, MeOH); HRMS (ESI-TOF) m/z: calcd for C₂₉H₃₅FNO₂ 448.2652 [M+H]; found 448.2639.

### Example 16: In-Vitro Cytotoxicity Assay

### A. Cytotoxicity of Cannabidiol Compounds Against Different Cancer Cell Line

All the ring annulated analogues of cannabinoid were evaluated for their cytotoxicity against number of human cancer cell lines. To evaluate the in-vitro cytotoxicity, a sulphorhodamine B (SRB) assay was carried out. SRB is a negatively charged pink aminoxanthine dye that binds to the basic amino acid of cells in mildly acidic conditions and is removed in basic conditions. For this, cells were grown in RPMI-1640 growth media. When cells reached 60-70% confluency, trypsinized and seeded in 96 well plates according to their inoculation density and incubated for 24 hours to attain their proper morphology. Next-day cells were again incubated for 48 hours with test compounds or paclitaxel (positive control). Next, cells were fixed with 50% of ice-cold TCA for 1 hours, washed with distilled water, and kept for air drying. Air-dried plates were then stained with 0.4% SRB dye for 30 minutes and subsequently with water and 1% v/v glacial acetic acid were then kept for air drying. SRB dye was dissolved by adding 100 μl of TRIS buffer (10 mM, pH 10.4); when the dye was dissolved, absorbance was taken in a plate reader at 540 nm. The viability of cells is measured based on bound dye after fixation, which is measured by absorbance at 540 nm.

$$i . Cell viability and growth inhibition were calculated by using the formula : - \% Cell Viability = \frac{Mean O . D . of treated cells}{Mean O . D . of control cell \times 100}$$

$$Growth Inhibition = 100 - cell viability$$

TABLE 1
Cytotoxicity of different Cannabidiol compounds against different cancer
cell line (conc. = 20 μM) Compound IIa is cannabidiol.

| | Sample | HCT- | | | Mia- | | | HEK- |
|---|---|---|---|---|---|---|---|---|
| S. No. | Code | 116 | MCF-7 | A549 | PaCa | PC3 | U251 | 293 |
| 1 | IIa | +++ | ++ | +++ | − | ++ | + | − |
| 2 | Ia | ++ | +++ | +++ | +++ | +++ | +++ | +++ |
| 3 | Ib | ++ | +++ | +++ | +++ | +++ | ++ | ++ |
| 4 | Ic | ++ | +++ | ++ | +++ | ++ | ++ | + |
| 5 | Id | +++ | +++ | +++ | +++ | +++ | ++ | + |
| 6 | Ie | +++ | +++ | + | + | + | + | + |
| 7 | If | ++ | +++ | +++ | +++ | +++ | +++ | ++ |
| 8 | Ig | ++ | ++ | ++ | + | ++ | + | + |
| 9 | Ih | ++ | +++ | ++ | +++ | +++ | ++ | ++ |
| 10 | Ii | ++ | +++ | +++ | +++ | ++ | + | + |
| 11 | Ij | + | + | + | + | + | + | + |
| 12 | Ik | + | + | + | + | + | + | + |
| 13 | Il | + | + | + | + | + | + | + |
| 14 | Im | + | + | + | + | + | + | + |
| 15 | In | ++ | + | ++ | + | ++ | + | + |
| 16 | Io | + | + | + | + | + | + | + |
| 17 | Paclitaxel | +++ | +++ | +++ | +++ | +++ | +++ | +++ |

In vitro cell inhibition at 20 M; + indicates less than 50% of inhibition (+<50% of inhibition), ++ indicates greater than 50-70% of inhibition (++>50-70% of inhibition) and +++ indicates greater than 70% of inhibition (+++>70% of inhibition).

### Results

The novel ring annulated analogues of CBD were screened for their antiproliferative activity against colorectal cancer, breast cancer, lung cancer, pancreatic cancer, prostate cancer, and glioblastoma cell lines such as HCT-116, MCF-7, A549, Mia-PaCa, PC-3 and U251, respectively and the results are presented in Table 1. Compound IIa showed greater than 70% in inhibition against HCT-116 and A549 cell lines. IIa also showed more than 50% of inhibition against MCF-7 and PC3 cell lines. Compound Ia showed greater than 70% inhibition against MCF-7, A549, Mia-PaCa, PC3, U251 and HEK-293 cell lines. Ia also showed more than 50% inhibition against HCT-116 cell lines. Compound Ib showed greater than 70% in inhibition against MCF-7, A549, Mia-PaCa and PC3 cell lines. Ib also showed more than 50% inhibition against HCT-116, U251 and HEK-293 cell lines. Compound Ic showed greater than 70% in inhibition against MCF-7 and Mia-PaCa cell lines. Ic also showed more than 50% inhibition against HCT-116, A549, PC3 and U251 cell lines. Compound Id showed greater than 70% inhibition against HCT-116, MCF-7, A549, Mia-PaCa and PC3 cell lines. Id also showed more than 50% inhibition against U251 cell lines. Compound Ie showed greater than 70% inhibition against HCT-116 and MCF-7 cell lines. Compound If showed greater than 70% inhibition against MCF-7, A549, Mia-PaCa, PC3 and U251 cell lines. If also showed more than 50% inhibition against HCT-116 and HEK-293 cell lines. Compound Ig showed more than 50% inhibition against HCT-116, MCF-7, A549 and PC3 cell lines. Compound Ih showed greater than 70% inhibition against MCF-7, Mia-PaCa and PC3 cell lines. Ih also showed more than 50% inhibition against HCT-116, A549, U251 and HEK-293 cell lines. Compound Ii showed greater than 70% inhibition against MCF-7, A549 and Mia-PaCa cell lines. Ii also showed more than 50% inhibition against HCT-116 and PC3 cell line. Compound In showed more than 50% inhibition against HCT-116, A549 and PC3 cell lines. Paclitaxel showed greater than 70% inhibition against HCT-116, MCF-7, A549, Mia-PaCa, PC3, U251 and HEK-293 cell lines.

### B: Inhibition Concentration (IC₅₀) of Different Cell Lines

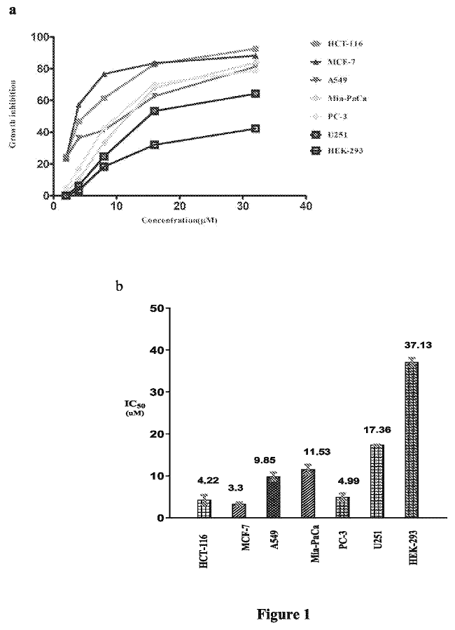

One of the generated analogues Id was taken for IC₅₀ determination using a concentration range from 2 μM to 128 μM against cancer cell lines. The compound Id had shown IC₅₀ values of 4.1 μM, 2.892 μM, 9.364 μM, 11.53 μM, 4.2 μM, and 17.36 μM against HCT-116, MCF-7, A549, Mia-PaCa-2, PC-3, and U251 cell lines, respectively (FIG. 1).

### Advantages of the Present Invention

The present invention has following advantages:
1. The present invention discloses the novel ring annulated analogues of CBD.
2. The present invention also discloses ring annulated analogues of CBD as a new anti-cancer agent.
3. The present invention also discloses the compounds with better activity in comparison to cannabidiol (CBD).
4. The present invention also discloses the new compound containing heterogeneous atoms which will help to improve the bioavailability.

1. A ring annulated cannabidiol analogue compound of Formula I 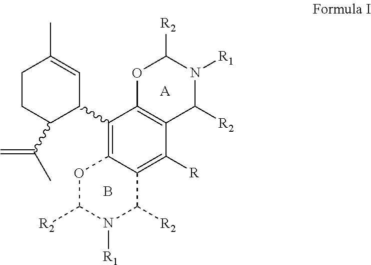
wherein, both A and B rings are simultaneously present or at least one of the rings is present;
R is alkyl;
R1 is independently selected from alkyl, aryl, heteroaryl, or cycloalkyl, wherein the alkyl or the aryl group is optionally substituted with one or more groups, independently selected from the group consisting of —F, alkyl, —O-haloalkyl, aryl, and heteroaryl; and
R2 is H.

2. The cannabidiol analogue as claimed in claim 1, wherein R1 is selected from the group consisting of
methyl, ethyl, n-propyl, n-butyl, n-hexyl,

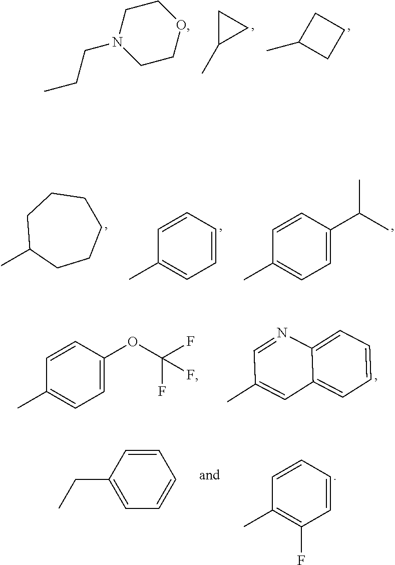

3. The cannabidiol analogue as claimed in claim 1, wherein the cannabidiol analogue is selected from the group consisting of (N-methyldihydrol-2H-oxazinyl)-cannabidiol, (N-ethyldihydrol-2H-oxazinyl)-cannabidiol, (N-propyldihydrol-2H-oxazinyl)-cannabidiol, (N-butyldihydrol-2H-oxazinyl)-cannabidiol, bis-{(N-butyldihydrol-2H-oxazinyl)}-cannabidiol, (N-hexyldihydrol-2H-oxazinyl)-cannabidiol, (N-morpholinopropyldihydrol-2H-oxazinyl)-cannabidiol, (N-cyclopropyldihydrol-2H-oxazinyl)-cannabidiol, (N-cyclobutyldihydrol-2H-oxazinyl)-cannabidiol, (N-cycloheptyldihydrol-2H-oxazinyl)-cannabidiol, (N-phenyldihydrol-2H-oxazinyl)-cannabidiol, (N-isopropylphenyldihydrol-2H-oxazinyl)-cannabidiol, (N-trifluoromethoxyphenyldihydrol-2H-oxazinyl)-cannabidiol, (N-quinolinedihydrol-2H-oxazinyl)-cannabidiol, (N-benzyldihydrol-2H-oxazinyl)-cannabidiol, and (N-2-fluorophenyldihydrol-2H-oxazinyl)-cannabidiol.

4. A process for the synthesis of ring annulated cannabidiol analogue compound of Formula I, 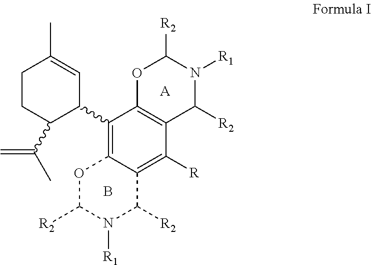
wherein, both A and B rings are simultaneously present or at least one of the ring is present;
R is alkyl;
R₁ is independently selected from alkyl, aryl, heteroaryl, or cycloalkyl; wherein the alkyl, aryl is optionally substituted with one or more groups, independently selected from the group consisting of —F, alkyl, —O-haloalkyl, aryl, and heterocyclyl; and
R2 is H
comprising the steps of:
i. coupling of fragment II and fragment III in the presence of an aldehyde IV at a temperature in the range of 25-30° C., for a period in the range of 13-16 hours optionally in presence of a solvent 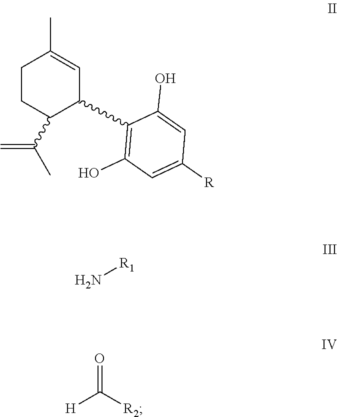
and
ii. purifying the obtained compound of formula I.

5. The process as claimed in claim 4, wherein the solvent is selected from the group consisting of dichloromethane, chloroform, isopropanol, acetone, acetonitrile methanol, and a combination thereof.

6. The process as claimed in claim 4, wherein compound of fragment III is selected from the group consisting of.

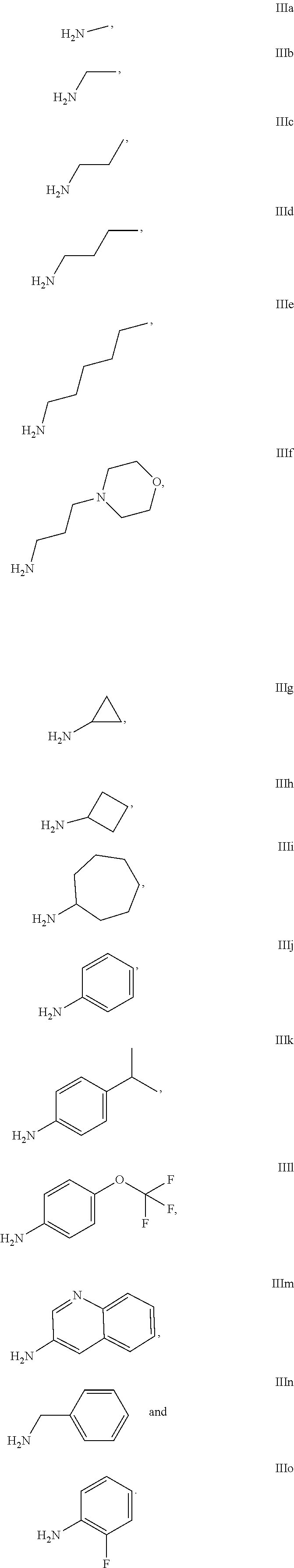

7. The process as claimed in claim 4, wherein the aldehyde IV is selected from the group consisting of formaldehyde, propionaldehyde, acetaldehydes, propionaldehyde, butyraldehyde, isovaleraldehyde, benzaldehyde and cinnamaldehyde.

8. The cannabidiol analogue as claimed in claim 1, wherein two stereo-centers are present which may be R and S as well as mixture of both.

9. A method for treating an anti-proliferative condition, comprising administering to a patient in need thereof an effective amount of the Formula I compound.

10. The method of claim 9, wherein the condition is selected from the group consisting of colorectal cancer, breast cancer, lung cancer, pancreatic cancer, prostate cancer, and brain cancer.
